(19) United States
(12) Patent Application Publication
(10) Pub. No.: US 2024/0100124 A1
(43) Pub. Date: Mar. 28, 2024

(54) Method of Treating Corneal Opacities and Scarring

(71) Applicant: University of Pittsburgh - Of the Commonwealth System of Higher Education, Pittsburgh, PA (US)

(72) Inventors: Bharesh Kumar Chauhan, Sewickley, PA (US); Kanwal Nischal, Pittsburgh, PA (US)

(21) Appl. No.: 18/274,317

(22) PCT Filed: Jan. 27, 2022
(86) PCT No.: PCT/US22/14019

§ 371 Date: Jul. 26, 2023

Related U.S. Application Data
(60) Provisional application No. 63/142,156, filed on Jan. 27, 2021.
(60) Provisional application No. 63/163,234, filed on Mar. 19, 2021.

(51) Int. Cl. A61K 38/18; A61K 9/00; A61K 38/17; C12N 15/113

(52) U.S. Cl. CPC A61K 38/18; A61K 9/0014; A61K 9/0048; A61K 38/1709; C12N 15/1137; C12N 2310/11; C12N 2310/14

(57) ABSTRACT

Provided herein are methods of treating corneal opacities in patients, such as neonates, including administering to the patient's eye or cornea a ZBTB7B and/or granulin polypeptide, and/or knocking down expression of HAS2 and/or HYAL1 in the patient's eye or cornea.

### CROSS REFERENCE TO RELATED APPLICATIONS

This application is the United States national phase of International Application No. PCT/US22/14019 filed Jan. 27, 2022, and claims priority to U.S. Provisional Patent Application No. 63/163,234 filed Mar. 19, 2021, and U.S. Provisional Patent Application No. 63/142,156 filed Jan. 27, 2021, the disclosures of each of which are hereby incorporated by reference in their entireties.

### STATEMENT REGARDING FEDERAL FUNDING

This invention was made with government support under Grant No. EY008098 awarded by the National Institutes of Health. The government has certain rights in the invention.

The Sequence Listing associated with this application was filed in electronic format via EFS-Web and is hereby incorporated by reference into the specification in its entirety. The name of the text file containing the Sequence Listing is 2108333_ST25.txt. The size of the text file is 36,428 bytes, and the text file was created on Jan. 26, 2022.

Congenital corneal opacities (CCO) are a group of blinding corneal disorders, where the underlying molecular mechanisms are poorly understood. They are rare corneal conditions with an incidence of about 2.2-3 per 100,000. It can be divided into primary and secondary corneal diseases, where the former are congenital and latter can either be congenital or acquired. Corneal transplantation is the standard treatment modality. However, they have a particularly high failure rate in the pediatric population due to complications such as rejection, infection, scar formation, or glaucoma. Alternative treatment modalities are therefore clearly needed through better understanding of CCO at the molecular level.

Diagnosis of CCO sub-types has significantly improved through imaging techniques. An early report found 40% of CCO diagnoses were incorrect when evaluated by ultrasound biomicroscopy (UBM) and that histopathological analysis would usually confirm the correct type identified by the imaging technique (Nischal, K. K., et al., Clinicopathological correlation of congenital corneal opacification using ultrasound biomicroscopy Br J Ophthalmol 86 (2002) 62-69). An example of the former comes from studies showing that sclerocornea (total corneal opacification) were correctly diagnosed by UBM to be Peters' anomaly, the most common CCO sub-type seen in the clinic.

Development of the cornea and molecular players underpinning CCO are poorly understood. Gene expression profiling is a technique that has been effective in identifying targets in the development of ocular tissues and congenital diseases in the lens, cornea, and retina. RNA-Seq is the most recent platform and is considered better than the usually employed microarrays, as it accurately quantifies RNA transcripts directly (Xu, J. et al., The FDA's Experience with Emerging Genomics Technologies-Past, Present, and Future AAPS J 18 (2016) 814-818 and Zhao, S., et al., Comparison of RNA-Seq and microarray in transcriptome profiling of activated T cells PLoS One 9 (2014) e78644). However, it has rarely been performed for pediatric tissues of ocular origin.

### SUMMARY

A method of treating corneal fibrosis in a patient is provided. The method comprising, reducing expression of HYAL1 and/or HAS2 in the eye of the patient.

According to another aspect or embodiment, a method of treating corneal fibrosis in a patient is provided. The method comprising, administering a granulin polypeptide and/or a ZBTB7B polypeptide to a patient's eye, in an amount effective to reduce corneal fibrosis in the patient.

According to yet another aspect or embodiment, a topical or parenteral pharmaceutical composition for delivery to the eye of a patient, comprising a granulin polypeptide and/or a ZBTB7B polypeptide, a nucleic acid encoding a granulin polypeptide and/or a nucleic acid encoding ZBTB7B polypeptide, an antisense or RNAi agent for knocking down expression of HYAL1 and/or HAS2 in the patient's eye or in the patient's cornea, and/or a nucleic acid encoding RNAi agent for knocking down expression of HYAL1 and/or HAS2 in the patient's eye or in the patient's cornea, and a pharmaceutically acceptable excipient or carrier is provided for use in a method of treating corneal fibrosis in a patient.

According to yet another aspect or embodiment, a topical or parenteral pharmaceutical composition for delivery to the eye of a patient is provided. The composition comprising a granulin polypeptide and/or a ZBTB7B polypeptide, a nucleic acid encoding a granulin polypeptide and/or a ZBTB7B polypeptide, an antisense or RNAi agent for knocking down expression of HYAL1 and/or HAS2 in the patient's eye or in the patient's cornea, and/or a nucleic acid encoding RNAi agent for knocking down expression of HYAL1 and/or HAS2 in the patient's eye or in the patient's cornea, and a pharmaceutically acceptable excipient or carrier.

The following numbered clauses describe various aspects or embodiments of the present invention.
Clause 1. A method of treating corneal fibrosis in a patient, comprising, reducing expression of HYAL1 and/or HAS2 in the eye of the patient.
Clause 2. The method of clause 1, comprising knocking down expression of HYAL1 and/or HAS2 in the eye of the patient.
Clause 3. The method of clause 2, comprising administering an RNAi agent to the eye of the patient in an amount effective to treat corneal opacity in the patient.
Clause 4. The method of clause 3, wherein the RNAi agent is an siRNA or an shRNA.
Clause 5. The method of clause 3, wherein the RNAi agent is administered to the patient's eye by administration to the patient's eye of a nucleic acid comprising a gene for expressing the RNAi agent, such as in a polyplex with a polycation.
Clause 6. The method of clause 2, comprising administering an antisense agent to the eye of the patient in an amount effective to reduce, treat, or prevent corneal opacity in the patient.
Clause 7. The method of any one of clauses 1-6, comprising knocking down HYAL1 in the eye of the patient.
Clause 8. The method of any one of clauses 1-6, comprising knocking down HAS2 in the eye of the patient.
Clause 9. The method of any one of clauses 1-6, comprising knocking down both HYAL1 and HAS2 in the eye of the patient.
Clause 10. A method of treating corneal fibrosis in a patient, comprising, administering a granulin polypeptide and/or a ZBTB7B polypeptide to a patient's eye, in an amount effective to reduce corneal fibrosis in the patient.
Clause 11. The method of clause 10, comprising administering a granulin polypeptide to a patient's eye, in an amount effective to reduce corneal opacity, corneal fibrosis, or corneal scarring in the patient.
Clause 12. The method of clause 11, wherein the granulin polypeptide comprises or consists of:
   the amino acid sequence of SEQ ID NO: 11,
   an amino acid sequence having at least 70, 80, 90, 95, 98, or 99% sequence identity or similarity to SEQ ID NO: 11, or
   an amino acid sequence comprising one or more granulin motifs, such as X₂₋₃CX₅₋₆CX₅CCX₈CCX₆CCX₅CCX₄CX₅₋₆CX₂ or X₃CX₆CX₅CCX₇CC, where each instance of X is, independently, any amino acid.
Clause 13. The method of clause 11, wherein the granulin polypeptide has the amino acid sequence of any one of SEQ ID NOS: 1-10 and 19-24, or a sequence having at least 70, 80, 90, 95, or 98% sequence identity or similarity to any one of SEQ ID NOS: 1-10, 19, or 20.
Clause 14. The method of clause 11, wherein the granulin polypeptide is a granulin 4, granulin 6, or granulin 7 polypeptide, or a combination of any of the preceding.
Clause 15. The method of any one of clauses 11-14, wherein a nucleic acid is administered to the patient's eye, comprising a gene for expressing the granulin polypeptide in the patient's eye, to produce the granulin polypeptide in the patient's eye.
Clause 16. The method of any one of clauses 11-14, wherein an mRNA encoding the granulin polypeptide is administered to the patient's eye to produce the granulin polypeptide in the patient's eye.
Clause 17. The method of clause 16, wherein the mRNA complexed into a lipid nanoparticle and the lipid nanoparticle comprising the mRNA is administered to the patient's eye.
Clause 18. The method of clause 15 or 16, wherein the nucleic acid or mRNA is complexed with a polycation, such as a polyethyleneimine (PEI), to form a polyplexed nucleic acid, which is administered to the patient's eye.
Clause 19. The method of clause 10, comprising administering a ZBTB7B polypeptide to a patient's eye, in an amount effective to reduce corneal opacity, corneal fibrosis, or corneal scarring in the patient.
Clause 20. The method of clause 19, wherein the ZBTB7B polypeptide has the amino acid sequence of SEQ ID NO: 13, or a sequence having at least 70, 80, 90, 95, 98, or 99% sequence identity or similarity to SEQ ID NO: 13.
Clause 21. The method of clause 19 or 20, wherein a nucleic acid is administered to the patient's eye, comprising a gene for expressing the ZBTB7B polypeptide in the patient's eye, to produce the ZBTB7B polypeptide in the patient's eye.
Clause 22. The method of clause 19 or 20, wherein an mRNA encoding the ZBTB7B polypeptide is administered to the patient's eye to produce the ZBTB7B polypeptide in the patient's eye.
Clause 23. The method of clause 22, wherein the mRNA complexed into a lipid nanoparticle and the lipid nanoparticle comprising the mRNA is administered to the patient's eye.
Clause 24. The method of clause 21 or 22, wherein the nucleic acid or mRNA is complexed with a polycation, such as a polyethyleneimine (PEI), to form a polyplexed nucleic acid, which is administered to the patient's eye.
Clause 25. The method of any one of clauses 10-24, comprising, administering a granulin polypeptide and/or a ZBTB7B polypeptide to the patient's cornea, and optionally to the patient's iris.
Clause 26. The method of any one of clauses 10-25, comprising administering both of a granulin polypeptide and/or a ZBTB7B polypeptide to the patient's eye.
Clause 27. The method of any one of clauses 1-26, comprising both administering a granulin polypeptide and/or a ZBTB7B polypeptide to the patient's eye or cornea, and knocking down expression of HYAL1 and/or HAS2 in the patient's eye or in the patient's cornea.
Clause 28. The method of any one of clauses 1-27, wherein the patient has a congenital corneal opacity.
Clause 29. The method of any one of clauses 1-27, wherein the patient has an acquired corneal opacity.
Clause 30. The method of clause 29, wherein the patient has corneal trauma.
Clause 31. The method of clause 29, wherein the patient has a corneal transplant and the transplanted cornea is treated.
Clause 32. The method of any one of clauses 1-31, wherein the patient has Fanconi anemia, Peters' anomaly (PA), sclerocornea, congenital hereditary endothelial dystrophy (CHED), congenital hereditary stromal dystrophy (CHSD), posterior polymorphous dystrophy (PPMD), congenital anterior staphyloma, granular corneal dystrophy, cystinosis, ichthyosis, trisomy 8 mosaicism, or Farber's disease.
Clause 33. The method of any one of clauses 1-32, wherein the patient is a human.
Clause 34. A topical or parenteral pharmaceutical composition for delivery to the eye of a patient, comprising a granulin polypeptide and/or a ZBTB7B polypeptide, a nucleic acid encoding a granulin polypeptide and/or a nucleic acid encoding ZBTB7B polypeptide, an antisense or RNAi agent for knocking down expression of HYAL1 and/or HAS2 in the patient's eye or in the patient's cornea, and/or a nucleic acid encoding RNAi agent for knocking down expression of HYAL1 and/or HAS2 in the patient's eye or in the patient's cornea, and a pharmaceutically acceptable excipient or carrier is provided for use in a method according to any one of clauses 1-33.
Clause 35. A topical or parenteral pharmaceutical composition for delivery to the eye of a patient, comprising a granulin polypeptide and/or a ZBTB7B polypeptide, a nucleic acid encoding a granulin polypeptide and/or a nucleic acid encoding ZBTB7B polypeptide, an antisense or RNAi agent for knocking down expression of HYAL1 and/or HAS2 in the patient's eye or in the patient's cornea, and/or a nucleic acid encoding RNAi agent for knocking down expression of HYAL1 and/or HAS2 in the patient's eye or in the patient's cornea, and a pharmaceutically acceptable excipient or carrier.

### BRIEF DESCRIPTION OF THE DRAWINGS

FIG. 1A provides an exemplary amino acid sequence of human progranulin (SEQ ID NO: 11), and FIG. 1B provides an exemplary mRNA sequence for human progranulin (SEQ ID NO: 12). FIG. 1C provides the amino acid sequence (SEQ ID NO: 19) of Atsttrin (Tang W, et al., The growth factor progranulin binds to TNF receptors and is therapeutic against inflammatory arthritis in mice. Science. 2011; 332(6028):478-484). FIG. 1D (reproduced from Dastpeyman M, et al., Structural Variants of a Liver Fluke Derived Granulin Peptide Potently Stimulate Wound Healing. J Med Chem. 2018 Oct. 11; 61(19):8746-8753) provides non-limiting examples of liver fluke (Opisthorchis viverrini) granulin polypeptides, including derivatives with amino acid substitutions (SEQ ID NOS: 20-24, top-to-bottom).

FIGS. 2A and 2B provide an exemplary amino acid sequence of human ZBTB7B (FIG. 2A, SEQ ID NO: 13, GenBank: AAH12070.1), and an exemplary mRNA sequence of human ZBTB7B (FIG. 2B, SEQ ID NO: 14, NM_001377453.1).

FIG. 3 provides an exemplary sequence for human HAS2 mRNA (SEQ ID NO: 15, U54804.1).

FIG. 4 provides an exemplary sequence for human HYAL1 mRNA (SEQ ID NO: 16, NCBI Reference Sequence: NR_047690.2).

FIG. 5. A-B, microcornea, peripheral scleralization, and corneal opacity in each of the right and left eyes; C-D, dehiscence of the Descemet membrane in the right eye (arrow) and central posterior segment irregularity in the left eye cornea by OCT, with hyper-reflective regions in both stroma demonstrating corneal haze; and E-G, iridiocorneal adhesions displayed in both eyes (arrows in F-G for left eye) by ultrasound biomicroscopy (UBM). H, Paraffin-embedded hematoxylin and eosin-stained section of the patient's cornea reveals disruption of the lamellar architecture and absence of Bowman's layer. There is central stromal thinning, and only peripheral segments of normal thickness Descemet's membrane are noted (arrowhead). I, Higher magnification image of the peripheral cornea reveals abnormal stromal architecture accompanied by blood vessels (arrows) as noted in the clinical photographs. Endothelial cells are only focally noted (arrowhead) and Descemet's membrane is attenuated and often difficult to discern. J, Periodic acid—Schiff staining reveals the presence of unusually thin Descemet's membrane (arrowheads) that is sometimes split (asterisk) or detached from the stroma.

FIGS. 6A and 6B. A, Comparative transcriptome variation analyses of the ACo transcriptome variation to the transcriptome variation of peripheral blood mononuclear cells from Fanconi anemia patients against healthy controls (PBMFA; GSE17233); B, the transcriptome variation from erythroid progenitor cells cultured from blood of beta-thalassaemia patients against healthy controls (EPCBT; GSE56088); and C, Venn diagram of misregulated genes from the ACo, PBMFA (F), and EPCBT (S) studies.

FIGS. 7A-7C. FIG. 7A, Expression data for ZBTB7B and GRN in ocular tissues of pediatric donor eye through RNASeq; FIG. 7B, ZBTB7B and GRN expression during early corneal development in the mouse from E10.5-E16.5 (GEO dataset: GSE121044); FIG. 7C, ZBTB7B and GRN expression in the mouse corneal epithelia from E14.5-2 yrs (GEO dataset: GSE43155). Abbreviations: Co, cornea; CoE, corneal epithelium; E, embryonic day; P, postnatal day; pNC, periocular neural crest; SK, stromal keratocytes. *represents p<0.05; **represents p<0.005.

FIGS. 8A-8E. FIG. 8A, Gene expression studies showing the levels in expression of ZBTB7B and important anti-inflammatory players together with FIG. 8B, GRN in epithelia of different organs of the mouse at late embryonic development (GEO dataset: GSE43381); FIG. 8C, fold-change of ZBTB7B in the lacrimal gland 8 hrs after chemical injury to mice corneas; FIG. 8D, transcriptome variation in ACo for ZBTB7B-dependent fibrotic factors SP1 and COL1A1; FIG. 8E, GRN fold-change in activated myofibroblasts upon addition of the wound-healing agent ethyl pyruvate. Abbreviations: Bla, bladder; Co, cornea; EP, ethyl pyruvate; Eso, esophagus; Lu, lung; Myofib, activated myofibroblasts; Sto, stomach; Tr, trachea; E, embryonic day. **represents p<0.005.

FIG. 9, provides Table 3, referenced below.

### DETAILED DESCRIPTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed aspects, whether provided in the examples or elsewhere herein.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more. Patent publications cited below are hereby incorporated herein by reference in their entirety to the extent of their technical disclosure and consistency with the present specification.

As used herein, the terms “comprising,” “comprise,” or “comprised,” and variations thereof, are open ended and do not exclude the presence of other elements not identified. In contrast, the term “consisting of” and variations thereof is intended to be closed and excludes additional elements in anything but trace amounts.

As used herein, the term “patient” or “subject” refers to members of the animal kingdom including but not limited to human beings and “mammal” refers to all mammals, including, but not limited to human beings.

As used herein, the “treatment” or “treating” of corneal opacity means administration to a patient by any suitable dosage regimen, procedure and/or administration route of a composition, device, or structure with the object of achieving a desirable clinical/medical end-point, including but not limited to, for a corneal opacity, reducing or preventing further development of corneal opacity, e.g., as determined below. An amount of any reagent or therapeutic agent, administered by any suitable route, effective to treat a patient is an amount capable of preventing, reducing, and/or eliminating corneal scarring. The therapeutically effective amount of each therapeutic may range from 1 pg per dose to 10 g per dose, including any amount there between, such as, without limitation, 1 ng, 1 μg, 1 mg, 10 mg, 100 mg, or 1 g per dose. The therapeutic agent may be administered by any effective route, but in the context of treatment of corneal scarring may be most typically delivered topically to the eye. The therapeutic agent may be administered as a single dose, at regular or irregular intervals, in amounts and intervals as dictated by any clinical parameter of a patient, or continuously.

Active ingredients, such as nucleic acids or analogs thereof, may be compounded or otherwise manufactured into a suitable composition for use, such as a pharmaceutical dosage form or drug product in which the compound is an active ingredient. Compositions may comprise a pharmaceutically acceptable carrier, or excipient. An excipient is an inactive substance used as a carrier for the active ingredients of a medication. Although “inactive”, excipients may facilitate and aid in increasing the delivery or bioavailability of an active ingredient in a drug product. Non-limiting examples of useful excipients include: anti-adherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts. In one example, a nucleic acid is delivered in a lipid nanoparticle.

Useful dosage forms include: intravenous, intramuscular, intraocular, or intraperitoneal solutions, oral tablets or liquids, topical ointments or creams, and transdermal devices (e.g., patches). In one embodiment, the compound is a topical liquid, emulsion, or lipid nanoparticle comprising a nucleic acid or analog thereof, such as an RNAi reagent, an mRNA, a DNA comprising a gene for expressing a polypeptide, or an antisense oligonucleotide.

Suitable dosage forms may include single-dose, or multiple-dose vials or other containers, such as medical syringes or droppers, containing a composition comprising an active ingredient useful for treatment of corneal opacity as described herein.

Pharmaceutical formulations adapted for administration include aqueous and non-aqueous sterile solutions which may contain, for example and without limitation, antioxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous solutions and suspensions may be prepared from sterile powders, granules and tablets.

Therapeutic compositions typically must be sterile and stable under the conditions of manufacture and storage. For example, sterile injectable solutions can be prepared by incorporating the active agent in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions in dosage unit form for ease of administration and uniformity of dosage. The specification for the dosage unit forms is dictated by and directly dependent on (a) the unique characteristics of the active compound and the particular therapeutic or prophylactic effect to be achieved, and (b) the limitations inherent in the art of compounding such an active compound for the treatment of sensitivity in individuals.

For corneal opacities, such as congenital or acquired corneal opacities, molecular therapies do not exist. The only treatment for these conditions is surgical intervention, namely corneal transplantation. Their success rates in the pediatric population are low due to complications ranging from graft rejection to insidious opacification. Thus, alternative treatments are clearly needed.

We found the zinc finger transcription factor ZBTB7B, which is known to prevent fibrosis by repressing the type I collagen gene, is highly expressed in the cornea compared to other tissues in the eye. We uncovered, unexpectedly, high expression of ZBTB7B in the cornea, lung, and trachea epithelia compared to bladder, esophagus, and stomach epithelia. Expression of several important anti-inflammatory players that prevent fibrosis, IL4, IL10, IL13, and TNF-alpha, were also shown to follow the same pattern. This suggested ZBTB7B are highly expressed in those tissues where anti-inflammatory responses are necessary. This led to the hypothesis that downregulation of the anti-fibrotic factor ZBTB7B in maldeveloped corneas leads to fibrosis and, subsequently, opacities of the cornea.

This was confirmed when we identified in a pediatric eye with CCO downregulation of ZBTB7B. In a separate study comparing scleroderma (fibrotic skin) fibroblasts to normal ones, where the fibrotic marker COL1A1 was shown to be upregulated upon ZBTB7B and Sp1 downregulation, we also found the same patterns of expression in the patient with CCO corneal transcriptome. This, again, confirmed downregulation of ZBTB7B, Sp1 and upregulation of COL1A1 may be responsible for corneal opacities in a maldeveloped cornea. Furthermore, from a microarray-based study on chemical burns (silver nitrate application) to mice cornea resulting in corneal opacities and blindness, the representative gene expression changes in the lacrimal gland post-injury compared to non-injured mice uncovered a significant downregulation in ZBTB7B. Based on all these findings, we propose that CCO can be prevented by addition of ZBTB7B.

We also uncovered the downregulation of an established wound-healing factor, GRN, in the cornea of a patient with CCO. Furthermore, in a study showing corneal wound-healing upon ethyl pyruvate addition to culture medium after TGFB1 treatment, which causes corneal keratocytes to form myofibroblasts, we identified a significant increase in GRN expression. This wound-healing response to TGFB1-induced fibrosis was shown to inhibit upregulation of profibrotic genes, thus, confirming GRN in the cornea may contribute to wound-healing. We also identified significant expression of GRN in the human cornea compared to other tissues in the eye, in addition to revealing elevated expression of the gene in epithelia of the lung and trachea compared to epithelia of the bladder, esophagus and stomach. The latter correlates with significant expression of several important anti-inflammatory players that prevent fibrosis, IL4, IL10, IL13, and TNF-alpha, therefore, suggesting GRN is highly expressed in tissues where anti-inflammatory responses would be fatal. Together, these data strongly suggest that GRN is a wound-healing factor in the cornea, and, therefore, addition of the factor to abnormally developing or injured corneas would be expected to prevent corneal scarring, resulting in blindness.

Lastly, HAS2 has been shown to be highly expressed in the corneal endothelium, in addition to the iris stroma. HAS2 (a known fibrotic marker) leads to formation of hyaluronic acid (HA), whereas the HYAL family (HYAL1-3) breaks down HA to form pro-inflammatory HA fragments. Overexpression of HA has been shown previously in vitro to mediate a fibrotic response in corneal keratocytes. When we performed RNA-seq analyses on opaque corneal tissue from a patient with FA and CCO against an age-matched normal cornea, HAS2 was significantly upregulated in addition to HYAL1. Both are important to scar formation, as HAS2 upregulates production of HA and HYAL1 degrades HA to form immunogenic HA fragments. HA fragments are well-known to elicit pro-inflammatory responses leading to scar formation, which in the cornea results in corneal opacification and, thus, blindness. Knock-down e.g., reduced or significant down-regulation of HAS2 or HYAL1 in the cornea through siRNA technologies is expected to prevent corneal opacification, and in the case of Peters' Anomaly, down-regulation of both in the cornea and iris stroma is expected to end the resulting corneal opacification.

These are non-stem cell approaches to preventing corneal scarring and molecular therapeutic approaches to surgical intervention (corneal transplantation) in the treatment of corneal opacity, e.g., CCO. The latter is particularly sought, as corneal transplants in the pediatric population have a poor outcome due to complications ranging from graft rejection to insidious opacification.

A corneal opacity is a loss of normal transparency. The normal cornea is transparent, with uniform collagen fibril distribution and spacing. Through various etiologies, irregularity and loss of transparency can occur, which may be referred to as “scarring”, though it may be more complex in nature because disruption of corneal basement membranes (Bowman's or Descemet membranes) could be involved. Clinical diagnosis of congenital corneal opacity classically may include: sclerocornea, tears in Descemet membrane, ulcers, metabolic, Peters anomaly, edema, and dermoid (STUMPED). An alternative classification of corneal opacities is based on whether they are primary versus secondary, or congenital versus acquired. Neonatal corneal opacities may be characterized as described in Nischal K K. A new approach to the classification of neonatal corneal opacities. Curr Opin Ophthalmol. 2012 September; 23(5):344-54. Corneal opacities may be congenital, or they may be acquired. Many different disorders may result in corneal opacifications of infancy, including developmental anomalies such as Peters' anomaly (PA), sclerocornea, congenital hereditary endothelial dystrophy (CHED), congenital hereditary stromal dystrophy (CHSD), posterior polymorphous dystrophy (PPMD), congenital anterior staphyloma, granular corneal dystrophy, cystinosis, ichthyosis, trisomy 8 mosaicism, and Farber's disease (See, e.g., Nischal K K. Genetics of Congenital Corneal Opacification—Impact on Diagnosis and Treatment. Cornea. 2015 October; 34 Suppl 10: S24-34).

Nucleic acids are presented in a 5′ to 3′ order, and amino acid sequences in an N-terminal to C-terminal order, unless otherwise described.

A “gene” is a sequence of DNA or RNA which codes for a molecule, such as a protein or a functional RNA, such as a non-coding RNA that has a function. Complementary refers to the ability of polynucleotides (nucleic acids) to hybridize to one another, forming inter-strand base pairs. Base pairs are formed by hydrogen bonding between nucleotide units in antiparallel polynucleotide strands. Complementary polynucleotide strands can base pair (hybridize) in the Watson-Crick manner (e.g., A to T, A to U, C to G), or in any other manner that allows for the formation of duplexes. When using RNA as opposed to DNA, uracil rather than thymine is the base that is complementary to adenosine. Two sequences comprising complementary sequences can hybridize if they form duplexes under specified conditions, such as in water, saline (e.g., normal saline, or 0.9% w/v saline) or phosphate-buffered saline), or under other stringency conditions, such as, for example and without limitation, 0.1×SSC (saline sodium citrate) to 10×SSC, where 1×SSC is 0.15M NaCl and 0.015M sodium citrate in water. Hybridization of complementary sequences is dictated, e.g., by salt concentration and temperature, with the melting temperature (Tm) lowering with increased mismatches and increased stringency. Perfectly matched sequences are said to be fully complementary, or have 100% sequence identity (gaps are not counted and the measurement is in relation to the shorter of the two sequences). A sequence that specifically hybridizes to another typically has at least 80%, 85%, 90%, 95%, or 99% sequence identity with the other sequence.

Gene expression is the process by which information from a gene is used in the synthesis of a functional gene product, e.g., a protein or functional RNA. Gene expression involves various steps, including transcription, translation, and post-translational modification of a protein, as is broadly-known.

By “expression” or “gene expression,” it is meant the processing of genetic information of a gene (without limitation, a functional genetic unit for producing a gene product, such as an RNA or a protein in a cell, or in other expression system encoded on a nucleic acid, and comprising: a transcriptional control sequence, such as a promoter and other cis-acting elements, such as transcriptional response elements (TREs) and/or enhancers; an expressed sequence that typically encodes a protein (referred to as an open-reading frame or ORF) or functional/structural RNA, and a polyadenylation sequence), to produce a gene product (typically a protein, optionally post-translationally modified or a functional/structural RNA). By “expression of genes under transcriptional control of,” or alternately “subject to control by,” a designated sequence such as TRE or transcription control element, it is meant gene expression from a gene containing the designated sequence operably linked (functionally attached, typically in cis) to the gene. A gene that is “under transcriptional control” of a TRE or transcription control element, is a gene that is transcribed at detectably different levels in the presence of a transcription factor.

A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when factors that regulate transcription, such as DNA-binding proteins, are present or absent—for example an amount of the respective inducer is available to the expression system (e.g., cell), or factors causing suppression of a gene are unavailable or displaced—effective to cause expression of the gene.

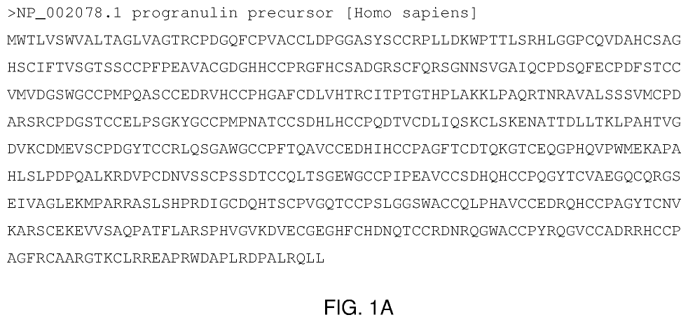

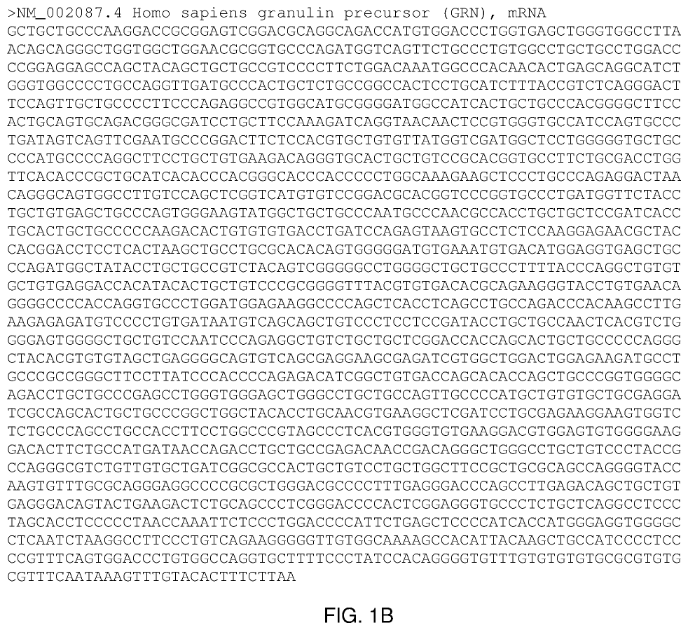

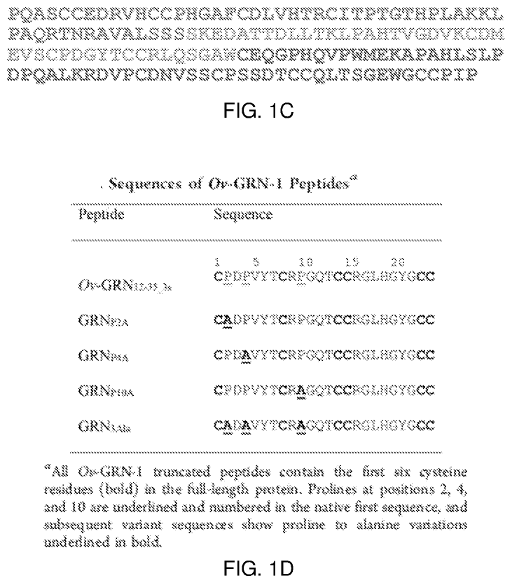

Granulin is the protein product of the GRN gene. Granulins are cleaved from progranulin (e.g., UniProtKB—P28799 (GRN_HUMAN) and NCBI Reference Sequence: NP_002078.1, see, FIG. 1A, noting that different protein isoforms and variants are identified; see also NCBI Reference Sequence: NM_002087.4, to form a number of active peptides, including Granulin A, Granulin B, Granulin C, etc. “Granulins” or “granulin polypeptides” include progranulin and various granulins, such as Granulin A, Granulin B, Granulin C, etc., as well as variants and derivatives thereof retaining granulin functionality in the context of the present disclosure. Individual granulins may have an approximate molecular weight of 6 kDa and may be structurally defined by the presence of 12 cysteines arranged in a characteristic motif: X₂₋₃CX₅₋₆CX₅CCX₈CCX₆CCX₅CCX₄CX₅₋₆CX₂ (SEQ ID NO: 25; see, e.g., Liu C J, Bosch X. Progranulin: a growth factor, a novel TNFR ligand and a drug target. Pharmacol Ther. 2012; 133(1):124-132) (numerical subscripts refer to the number of amino acids referenced, such that X₂₋₃ refers to either XX or XXX, where X is any natural amino acid) or X₃CX₆CX₅CCX₇CC (SEQ ID NO: 26; Dastpeyman M, et al., Structural Variants of a Liver Fluke Derived Granulin Peptide Potently Stimulate Wound Healing. J Med Chem. 2018 Oct. 11; 61(19):8746-8753), where each instance of X is, independently, any amino acid.

Full length progranulin, or a granulin polypeptide may be delivered to the eye for therapeutic effect Likewise progranulin- or granulin-encoding mRNA may be delivered to the eye either in the form of an mRNA reagent, or in the form of a gene for expression of the mRNA. If a gene for expressing an mRNA encoding a granulin (including progranulin as a class) is to be delivered to the eye, a nucleic acid comprising the gene may be delivered as naked DNA, with or without a suitable vector, such as a plasmid or viral vector (e.g., Adenovirus, Adeno-associated virus, herpesvirus, γ-retrovirus, etc.), as are known and are amply described in the literature, with many cloning and expression systems being described and commercially-available. Due to the potential of toxicity and immunogenicity viral vectors may be disfavored even though they may have higher transfection efficiencies. Although alone, naked DNA or mRNA may not result in particularly high transfection efficiencies, they may be complexed with polycationic compositions, such as chitosan or polyethyleneimine, to form complexes, referred to polyplexes (see, e.g., Midoux P, et al., Polymer-based gene delivery: a current review on the uptake and intracellular trafficking of polyplexes. Curr Gene Ther. 2008 October; 8(5):335-52; Wilson D R, et al., The role of assembly parameters on polyplex poly (beta-amino ester) nanoparticle transfections. Biotechnol Bioeng. 2019 May; 116(5):1220-1230; and Godbey W T, et al., Tracking the intracellular path of poly(ethylenimine)/DNA complexes for gene delivery. Proc Natl Acad Sci USA. 1999 Apr. 27; 96(9):5177-81).

Polypeptides may be engineered granulins comprising (e.g., fused with, in-frame) one or more granulin motifs, optionally combined with motifs not obtained from a granulin, or with native granulins having one or more amino acid substitutions. An exemplary engineered granulin polypeptide is Atsttrin, combining half units of granulins A, C, and F plus linkers (FIG. 1C, see, e.g., Liu C J, Bosch X., Pharmacol Ther. 2012; 133(1):124-132)

Non-human granulin polypeptides may perform equally well in the methods described herein. For example liver fluke (Opisthorchis viverrini, “Ov granulin polypeptides”) or carp granulin polypeptides may be used in the methods provided herein. Dastpeyman M, et al., (J Med Chem. 2018 Oct. 11; 61(19):8746-8753) provides sequences of Ov granulin polypeptides, sequence-modified versions of those polypeptides having certain Pro-to-Ala substitutions, including those depicted in FIG. 1D. Of note, modified Ov granulin polypeptides, such as GR_{NP4A} of Dastpeyman et al., (See, FIG. 1D) were shown to be functional in wound healing, if not superior to native Ov granulin polypeptides, and are expected to be functional in the methods provided herein.

Further exemplary granulin polypeptides are disclosed in U.S Pat. No. 9,655,947, providing a peptide having an amino acid sequence of a granulin (e.g., SEQ ID NOS: 11-20 of U.S. Pat. No. 9,655,947, noting that SEQ ID NO: 11 of that document is, like the sequence of FIG. 1A of the present document, a progranulin) or a sequence having 95% sequence identity to that sequence, which is described as being useful for treatment of neuropathic pain by delivery to a neuron. International Patent Publication No. WO 2018/013775 provides non-limiting examples of additional granulin polypeptide sequences, such as:
granulin-1 or a variant with greater than 70, 80, 90, 95, or 98%>sequence identity or similarity to

(SEQ ID NO: 1)
GGPCQVDAHCSAGHSCIFTVSGTSSCCPFPEAVACGDGHHCCPRGFHCS;
ADGRSCFQ

granulin-2 or a variant with greater than 70, 80, 90, 95, or 98%>sequence identity or similarity to

(SEQ ID NO: 2)
AIQCPDSQFECPDFSTCCVMVDGSWGCCPMPQASCCEDRVHCCPHGAFC;
DLVHTRCIT

granulin-3 or a variant with greater than 70, 80, 90, 95, or 98%>sequence identity or similarity to,

(SEQ ID NO: 3)
VMCPDARSRCPDGSTCCELPSGKYGCCPMPNATCCSDHLHCCPQDTVCDL;
IQSKCLS

granulin-4 or a variant with greater than 70, 80, 90, 95, or 98% sequence identity or similarity to,

(SEQ ID NO: 4)
CDMEVSCPDGYTCCRLQSGAWGCCPFTQAVCCEDHIHCCPAGFTCDTQKG
TCEQ
(also, DVKCDMEVSCPDGYTCCRLQSGAWGCCPFTQAVCCEDHIHCCP
AGFTCDTQKGTCE (SEQ ID NO: 5));

granulin-5 or a variant with greater than 70, 80, 90, 95, or 98% sequence identity or similarity to,

(SEQ ID NO: 6)
DVPCDNVSSCPSSDTCCQLTSGEWGCCPIPEAVCCSDHQHCCPQGYTCVA
EGQCQR;

granulin-6 or a variant with greater than 70, 80, 90, 95, or 98% sequence identity or similarity to,

(SEQ ID NO: 7)
DIGCDQHTSCPVGQTCCPSLGGSWACCQLPHAVCCEDROHCCPAGYTCNV
KARSCEK
(also, IGCDQHTSCPVGQTCCPSLGGSWACCQLPHAVCCEDRQHCCPA
GYTCNVKARSCE (SEQ ID NO: 8));

granulin-7 or a variant with greater than 70, 80, 90, 95, or 98% sequence identity or similarity to,

(SEQ ID NO: 9)
DVECGEGHFCHDNQTCCRDNRQGWACCPYRQGVCCADRRHCCPAGFRCAA
RGTKCL;

and
paragranulin or a variant with greater than 70, 80, 90, 95, or 98% sequence identity or similarity to, TRCPDGQFCPVACCLDPGGASYSCCRPLLD (SEQ ID NO: 10). U.S. Patent Application Publication No. 2021/0008163 A1 and International Patent Application Publication No. WO 2016/125330 A1, incorporated by reference for their technical disclosures, provide additional examples of granulin peptides.

A recombinant Adeno-associated viral vector, serotype 1 carrying a gene encoding GRN (designated PBFT02) is currently being assessed as a gene therapy for frontotemporal dementia.

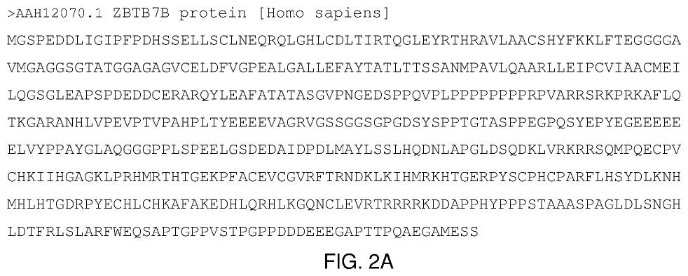

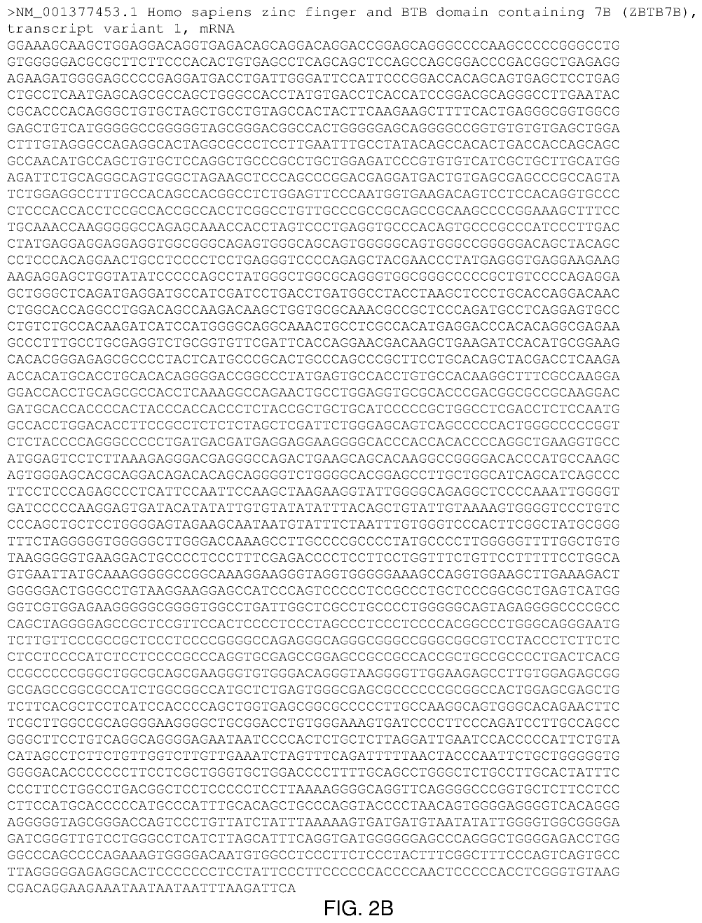

The granulin or ZBTB7B polypeptides may have 100% sequence identity with a natural polypeptide, e.g., as is shown for granulin in FIG. 1A or for ZBTB7B in FIG. 2A. The amino acid sequence of any described polypeptide may be modified yet still retain functionality as described herein, by the addition, deletion, or conservative substitution of one or more amino acids of the polypeptide. Hybrid, or chimeric polypeptides may be provided, comprising a natural or base-modified amino acid granulin or ZBTB7B polypeptide sequence, joined in-frame with a different polypeptide sequence, such as a signal sequence, a tag for use in purification, a targeting ligand sequence, or an active moiety, such as one of more additional iterations of the same or different granulin or ZBTB7B polypeptide sequence(s).

ZBTB7B refers to the “zinc finger and BTB domain containing 7B” protein, e.g., Gene ID: 51043 (describing various isoforms of ZBTB7B). FIGS. 2A and 2B provide exemplary amino acid and mRNA sequences for human ZBTB7B. Useful amino acid sequences for ZBTB7B include the sequence of FIG. 2A and a variant thereof with greater than 70, 80, 90, 95, or 98% sequence identity or similarity to the amino acid sequence of FIG. 2A, and DNA or mRNAs having a coding sequences (ORF) encoding an amino acid of FIG. 2A, or a variant thereof with greater than 70, 80, 90, 95, or 98% sequence identity or similarity to the amino acid sequence of FIG. 2A.

The granulin or ZBTB7B polypeptides, such as optimized hybrid subunits of granulin or ZBTB7B, e.g., nanoparticles containing granulin or ZBTB7B polypeptides, such as optimized hybrid subunits of granulin or ZBTB7B, as described herein may be delivered to the cornea as a protein, or in the form of a nucleic acid encoding the therapeutic peptide for expression in appropriate cell population(s). The polypeptide, and any other reagent described herein (e.g., RNAi agents or antisense agents described below), may be delivered topically to the eye, e.g., to the cornea, or injected into appropriate anatomical features or structures of the eye, e.g., the aqueous humor, for example as drops, emulsions, suspensions, gels (e.g., in situ gelling), micelles, nanoparticles, nanosuspensions, liposomes, dendrimers, contact lenses, implants, DNA or nucleic acid polyplexes, or microneedles (see, e.g., Patel A, et al., Ocular drug delivery systems: An overview. World J Pharmacol. 2013; 2(2):47-64). The unique nature of the proteins and nucleic acid reagents described herein will determine the ultimate, optimal, dosage form for effective delivery of the therapeutic agent to the cells, and particular anatomical structure of the eye, which can be determined by a person of skill in the drug delivery arts. For example, formulations for delivery of granulin are already developed and can be adapted for ocular/corneal delivery.

It should be noted that any DNA coding sequence, or mRNA coding sequence for production of a polypeptide as described herein, e.g., a granulin or a ZBTB7B polypeptide, such as optimized hybrid subunits of granulin or ZBTB7B, e.g., nanoparticles containing granulin or ZBTB7B polypeptides, such as optimized hybrid subunits of granulin or ZBTB7B, may be codon-optimized for expression in a particular cell, tissue, or organism, depending on its final use, for example in a human cornea for direct transfection of corneal cells.

Polypeptides (e.g., proteins) described herein for delivery as a therapeutic agent, e.g., a granulin or ZBTB7B polypeptide, such as optimized hybrid subunits of granulin or ZBTB7B, e.g., nanoparticles containing granulin or ZBTB7B polypeptides, such as optimized hybrid subunits of granulin or ZBTB7B, may be prepared in suitable cells (e.g., bacterium, yeast, insect cells, or mammalian cells), for in vitro production, and coding sequences may be codon-optimized for production in that system. Alternatively a polypeptide may be chemically synthesized or synthesized in a cell-free system, such as a cell extract. A person of skill in the art of protein synthesis and/or recombinant genetics may prepare a given polypeptide

If provided as a nucleic acid comprising a gene encoding the granulin or ZBTB7B polypeptide, such as optimized hybrid subunits of granulin or ZBTB7B, e.g., nanoparticles containing granulin or ZBTB7B polypeptides, such as optimized hybrid subunits of granulin or ZBTB7B, the nucleic acid may be prepared by any suitable means, such as a linear DNA strand, a plasmid or other extrachromosomal nucleic acid molecule, or as an mRNA. Depending on their size, polypeptides and nucleic acids may be prepared synthetically, by any suitable method, such as solid-phase synthesis, PCR, in vitro transcription (see, e.g., Pardi N, et al., mRNA vaccines—a new era in vaccinology. Nat Rev Drug Discov. 2018 April; 17(4):261-279), or primer extension based on a suitable template (e.g., DNA).

For knocking down expression of a gene, RNA levels in a cell, e.g., mRNA levels, can be controlled post-transcriptionally. Native mechanisms, including: endogenous gene silencing mechanisms, interference with translational mechanisms, interference with RNA splicing mechanisms, and destruction of duplexed RNA by RNAse H, or RNAse H-like activity. As is broadly-recognized by those of ordinary skill in the art, these endogenous mechanisms can be exploited to decrease or silence mRNA activity in a cell or organism in a sequence-specific, targeted manner. Antisense technology typically involves administration of a single-stranded antisense oligonucleotide (ASO) that is chemically modified, e.g., as locked nucleic acid or gapped-locked nucleic acid, for bio-stability, and is administered in sufficient amounts to effectively penetrate the cell and bind in sufficient quantities to target mRNAs in cells. RNA interference (RNAi) harnesses an endogenous and catalytic gene silencing mechanism, which means that once, e.g., a microRNA, or double-stranded siRNA has been delivered into the cytosol, they are efficiently recognized and stably incorporated into the RNA-induced silencing complex (RiSC) to achieve prolonged gene silencing. Either antisense technology or RNAi may be used effectively to knock-down or silence expression of a gene or gene product, such as HAS2 or HYAL1 (see, e.g., Watts J K, et al., Silencing disease genes in the laboratory and the clinic. J Pathol. 2012; 226(2):365-379. doi:10.1002/path.2993). It should be noted that siRNAs and/or ASOs targeting HAS2 or HYAL1 are commercially available from sources such as Horizon™ or Thermo Fisher Scientific, among many other sources, or are readily determined and synthesized based on broadly-known algorithms and calculators using an appropriate mRNA/cDNA sequence as input, such as, without limitation, SEQ ID NOS: 15 or 16, or the variants thereof. One siRNA, or multiple siRNAs targeting different portions of a target mRNA may be used.

The terms “iRNA,” “RNAi agent,” “RNAi agent,” and “RNA interference agent” as used interchangeably herein, refer to an agent that contains RNA nucleotides, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., knocks down or silences, the expression of HAS2 or HYAL1 RNA in a cell, e.g., a cell within a subject, such as a mammalian subject.

In one aspect, an RNAi agent includes a single stranded RNAi that interacts with a target RNA sequence, e.g., an HAS2 or HYAL1 RNA sequence, to direct the cleavage of the target RNA. Without wishing to be bound by theory it is believed that long double stranded RNA introduced into cells is broken down into double stranded short interfering RNAs (siRNAs) comprising a sense strand and an antisense strand by a Type III endonuclease known as Dicer. Dicer, a ribonuclease-III-like enzyme, processes these dsRNA into 19-23 base pair short interfering RNAs with characteristic two base 3′ overhangs. These siRNAs are then incorporated into an RNA-induced silencing complex (RISC) where one or more helicases unwind the siRNA duplex, enabling the complementary antisense strand to guide target recognition. Upon binding to the appropriate target mRNA, one or more endonucleases within the RISC cleave the target to induce silencing. Thus, in one aspect an RNAi is a single stranded RNA (ssRNA) (the antisense strand of an siRNA duplex) generated within a cell and which promotes the formation of a RISC complex to effect silencing of the target gene. Accordingly, the term “siRNA” is also used herein to refer to an interfering RNA (iRNA).

In another aspect, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., Single-stranded siRNAs activate RNAi in animals. (2012) Cell 150:883-894.

In another aspect, an “iRNA” or RNAi agent” for use in the compositions and methods described herein is a double stranded RNA and can be referred to herein as a “double stranded RNAi agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, e.g., an HAS2 or HYAL1 RNA. In some aspects, a double stranded RNA (dsRNA) triggers the degradation of a target RNA, e.g., an mRNA, through a post-transcriptional gene-silencing mechanism referred to herein as RNA interference or RNAi.

The majority of nucleotides of each strand of a dsRNA molecule may be ribonucleotides, but as described in detail herein, each or both strands can also include nucleotide analogs, where one or more non-ribonucleotides, e.g., a deoxyribonucleotide and/or a modified nucleotide. In addition, as used in this specification, an “RNAi agent” or “RNAi agent” may include ribonucleotides with chemical modifications; an RNAi agent may include substantial modifications at multiple nucleotides. As used herein, the term “modified nucleotide” refers to a nucleotide having, independently, a modified sugar moiety, a modified inter-nucleotide linkage, and/or modified nucleobase. Thus, the term modified nucleotide encompasses substitutions, additions or removal of, e.g., a functional group or atom, to inter-nucleoside linkages, sugar moieties, or nucleobases. The modifications suitable for use in the agents described herein include all types of modifications disclosed herein or known in the art. Any such modifications, as used in a siRNA type molecule, are encompassed by “RNAi agent” or “RNAi reagent” for the purposes of this disclosure.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some aspects, the hairpin loop can comprise at least 2, at least 3, at least 4, at least 5, at least 6, at least 7, at least 8, at least 9, at least 10, at least 20, at least 23, or more unpaired nucleotides. In some aspects, the hairpin loop can be 10 or fewer nucleotides. In some aspects, the hairpin loop can be 8 or fewer unpaired nucleotides. In some aspects, the hairpin loop can be 4-10 unpaired nucleotides. In some aspects, the hairpin loop can be 4-8 nucleotides.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In one aspect, an RNAi agent is a dsRNA, each strand of which comprises 19-23 nucleotides, that interacts with a target RNA sequence, e.g., an HAS2 or HYAL1 RNA, without wishing to be bound by theory, long double stranded RNA introduced into cells is broken down into siRNA by a Type III endonuclease known as Dicer. Dicer, a ribonuclease-III-like enzyme, processes the dsRNA into 19-23 base pair short interfering RNAs with characteristic two base 3′ overhangs. The siRNAs are then incorporated into an RNA-induced silencing complex (RISC) where one or more helicases unwind the siRNA duplex, enabling the complementary antisense strand to guide target recognition. Upon binding to the appropriate target RNA, one or more endonucleases within the RISC cleave the target to induce silencing. In one aspect, an RNAi agent is a dsRNA of 24-30 nucleotides that interacts with a target RNA sequence, e.g., an HAS2 or HYAL1 RNA sequence, to direct the cleavage of the target RNA.

“Complementary” sequences, as used herein, can also include, or be formed entirely from, non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary”, “fully complementary”, and “substantially complementary” herein can be used with respect to the base matching between the sense strand and the antisense strand of a dsRNA, or between the antisense strand of an RNAi agent and a target sequence, as will be understood from the context of their use.

As used herein, a polynucleotide that is “substantially complementary to at least part of a messenger RNA (mRNA)” refers to a polynucleotide that is substantially complementary to a contiguous portion of the mRNA of interest (e.g., an HAS2 or HYAL1 RNA).

Accordingly, in some aspects, the antisense strand polynucleotides disclosed herein are fully complementary to the target HAS2 or HYAL1 RNA sequence. In other aspects, the antisense strand polynucleotides disclosed herein are substantially complementary to the target HAS2 or HYAL1 RNA sequence and comprise a contiguous nucleotide sequence which has at least about 80% sequence identity to the nucleotide sequence of any of SEQ ID NOS: 15 or 16, or a fragment thereof, such as about 85%, about 86%, about 87%, about 88%, about 89%, about 90%, about 91%, about 92%, about 93%, about 94%, about 95%, about 96%, about 97%, about 98%, or about 99% complementary.

It is understood that the sequence of the HAS2 or HYAL1 RNA must be sufficiently complementary to the antisense strand of the RNAi agent for the agent to be used in the indicated patient, e.g. human, mammalian, or vertebrate species.

The term “inhibiting”, as used herein, is used interchangeably with “reducing”, “silencing”, “downregulating”, “suppressing”, “knocking down”, and other similar terms, and includes any level of inhibition.

The phrase “knocking down (or silencing) of HAS2 or HYAL1 RNA,” as used herein, includes inhibition of expression of any HAS2 or HYAL1 gene (such as, e.g., a mouse HAS2 or HYAL1 gene, a rat HAS2 or HYAL1 gene, a monkey HAS2 or HYAL1 gene, or a human HAS2 or HYAL1 S2 gene) as well as variants or mutants of an HAS2 or HYAL1 gene, in its production of HAS2 or HYAL1 RNA, affecting the stability of HAS2 or HYAL1 RNA, such as by antisense or RNAi technologies. “Knocking down (or silencing) of HAS2 or HYAL1 RNA” includes any level of inhibition of an HAS2 or HYAL1 RNA, e.g., at least partial suppression of the expression of an HAS2 or HYAL1 RNA, such as an inhibition by at least about 20%. In certain aspects, inhibition is by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

The expression of an HAS2 or HYAL1 RNA may be assessed based on the level of any variable associated with HAS2 or HYAL1 RNA expression, e.g., HAS2 or HYAL1 RNA level. The expression of an HAS2 or HYAL1 RNA may also be assessed indirectly based on assay of physiological markers associated with decreased expression of the HAS2 or HYAL1 RNA in a patient.

In one aspect, at least partial suppression of the expression of an HAS2 or HYAL1 RNA, is assessed by a reduction of the amount of HAS2 or HYAL1 RNA that can be isolated from or detected in a cell or group of cells, e.g., in a corneal cell. A reduction of the amount of HAS2 or HYAL1 RNA in a cell or tissue in which an HAS2 or HYAL1 gene is transcribed and which has been treated such that the expression of an HAS2 or HYAL1 RNA is inhibited, may be determined as compared to a second cell or tissue substantially identical to the first cell or tissue but which has not been so treated (control cells), e.g., obtained and cultured from a biopsy. The degree of inhibition may be expressed in terms of:

$$\frac{( mRNA in control cells ) - ( mRNA in treated cells )}{( mRNA in control cells )} \times 100 \% )$$

The phrase “contacting a cell with an RNAi agent,” such as a dsRNA, as used herein, includes contacting a cell by any possible means. Contacting a cell with an RNAi agent includes contacting a cell in vitro with the iRNA or contacting a cell in vivo with the iRNA. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell. Further, an shRNA RNAi agent can be produced from a gene for expressing an shRNA, transferred by any suitable means, such as by recombinant vector such as a recombinant Adeno-associated virus (AAV) or retrovirus vector, or by gene editing, such as by CRISPR-Cas or TALENS methods, as are broadly-known. These technologies are broadly-known by those of ordinary skill and resources, such as suitable vectors and production systems are broadly-available, including from commercial sources.

Contacting a cell in vitro may be done, for example, by incubating the cell with the RNAi agent. Contacting a cell in vivo may be done, for example, by injecting or placing the RNAi agent into or near the tissue where the cell is located, such as a tumor, or by injecting the RNAi agent into another area, e.g., the bloodstream or the subcutaneous space, such that the agent will subsequently reach the tissue where the cell to be contacted is located. For example, the RNAi agent may contain and/or be coupled to a ligand, e.g., GalNAc3, which directs the RNAi agent to a site of interest, e.g., the liver. Combinations of in vitro and in vivo methods of contacting are also possible. For example, a cell may also be contacted in vitro with an RNAi agent and subsequently transplanted into a subject.

In one aspect, contacting a cell with an iRNA includes “introducing” or “delivering the iRNA into the cell” by facilitating or effecting uptake or absorption into the cell. Absorption or uptake of an iRNA can occur through unaided diffusive or active cellular processes, or by use of auxiliary agents or devices. Introducing an iRNA into a cell may be in vitro and/or in vivo. For example, for in vivo introduction, an iRNA can be injected into a tissue site or administered systemically. In vitro introduction into a cell includes methods known in the art such as electroporation and lipofection. Further approaches are known in the art.

As used herein, and further to the discussion above regarding an iRNA reagents, “agent” or “RNAi agent”, when used in the context of an antisense, RNAi, or ribozyme, or other single-stranded or double-stranded RNA interfering nucleic acids, refers not only to RNA structures, but effective nucleic acid analog structures. In antisense and RNAi technologies, use of RNA poses significant delivery issues due to the lability of RNA molecules. As such, RNA is commonly chemically modified to produce nucleic acid analogs, not only to enhance stability of the nucleic acid molecules, but often resulting in increased binding affinity, and with reduced toxicity. Such modifications are broadly-known to those of ordinary skill in the art, and are available commercially (see, e.g., Corey, D. R., Chemical modification: the key to clinical application of RNA interference? (2007) J Clin Invest., 117(12):3615-3622, also describing RNAi, and U.S. Patent Application Publication No. 2017/0081667, incorporated herein by reference for its technical disclosure). Non-limiting examples of modifications to the nucleic acid structure in nucleic acid analogs include: modifications to the phosphate linkage, such as phosphoramidates or phosphorothioates; sugar modification, such as 2′-O, 4′-C methylene bridged, locked nucleic acid (LNA), 2′-methoxy, 2′-O-methoxyethyl (MOE), 2′-fluoro, S-constrained-ethyl (cEt), and tricyclo-DNA (tc-DNA); and non-ribose structures, such as phosphorodiamidate morpholino (PMO) and peptide-nucleic acids (PNA).

In addition to those HAS2- or HYAL1-active RNAi agents described herein, antisense agents (ASOs), other RNAi agents, ribozyme agents, and other nucleic acid-based methods of reducing gene expression, can be designed and tested based on known sequences of HAS2 or HYAL1 RNAs and gene structure (exemplary sequences are provided herein). Based on the present disclosure, one of ordinary skill can design, and/or produce an active agent capable of knocking down HAS2 or HYAL1 expression. Of note, a number of publications describe algorithms for generating candidate iRNA sequences, and publicly available software can be used to implement those algorithms. As such, typically, one only needs to enter an mRNA sequence into a calculator to produce candidate iRNAs.

As above, RNAi reagents, such as an siRNA, may have 100% sequence identity with a portion or fragment of any one or more of SEQ ID NOS: 15 or 16, or a sequence complementary thereto, or may include one or more additional nucleobases at their 3′ or 5′ end, or may include one or more substitutions that do not substantially interfere with the activity of the RNAi agent in knocking down or silencing HAS2 or HYAL1 expression. Also, SEQ ID NOS: 15 or 16 are exemplary mRNAs of HAS2 or HYAL1. Alleles, mutations, or other variants or polymorphisms (e.g., single-nucleotide polymorphisms, SNPs) of HAS2 or HYAL1 sequences are possible, and as such effective agents, such as RNAi and antisense agents may be substituted to accommodate those variants. Further, some sequence mismatches in RNAi agents are not only tolerated, but may be beneficial (see, e.g., Wu, H. et al., “Improved siRNA/shRNA Functionality by Mismatched Duplex” PLoS One. 2011; 6(12): e28580). As such, sequences having up to 90% or 95% (two or one mismatches, respectively) sequence identity with SEQ ID NOS: 15 or 16 are expected, in many circumstances, to be effective RNAi agents.

In aspects, a useful antisense oligonucleotide, e.g., a nucleic acid or nucleic acid analog, comprises a sequence having at least 90% sequence identity, at least 95% sequence identity, or 100% sequence identity with one of SEQ ID NOS: 15 or 16. In aspects, the antisense oligonucleotide is an LNA.

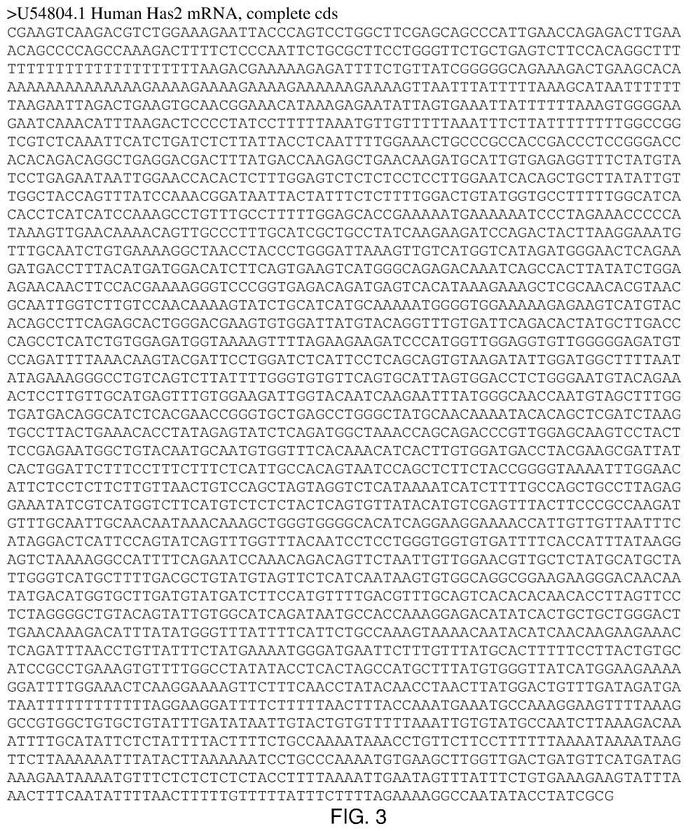

As described above, design and implementation of interfering RNA and antisense reagents useful in knocking down expression of a target gene, such as HAS2 or HYAL1 are well within the skill of an ordinary artisan, with commercial sources and design methods being broadly-available. siRNA targeting specific mRNAs are broadly-available commercially, and methods of determining and testing potential siRNA candidates are broadly-available, commercial, and otherwise (see, e.g., Tuschl T. Expanding small RNA interference. Nature Biotech 2002; 20:446-8 and Hu B, et al., Therapeutic siRNA: state of the art. Signal Transduct Target Ther. 2020 Jun. 19; 5(1):101). These references provide a roadmap as to how to design, make, and use RNAi (RNA interference) reagents, including nucleic acid modifications, shRNA delivery systems and vectors, and pharmaceutical formulations, such as lipid nanoparticles. See, for example, Li Y, et al., Silencing of hyaluronan synthase 2 suppresses the malignant phenotype of invasive breast cancer cells. Int J Cancer. 2007 Jun. 15; 120(12):2557-67 (“Four siRNA duplexes designed with symetric 30TT overhangs to target different nucleotide sequences (no 1, 1043-1061; no 2,1402-1420; no 3, 1617-1637; no 4, 1671-1691 of the humanHAS2 gene (Genbank accession number U54804, see FIG. 3) were obtained from Qiagen, UK”, p. 2558, referencing sequence numbering in Genbank Accession No, U54804.1 represented in FIG. 3 (SEQ ID NO: 15, see, also, exemplary amino acid sequence provided in GenBank: AAC50692.1)) left, all were shown to affect Hyaluronan and HAS2 mRNA expression; see also Zhang H, -Y, Liang F, Wang F, Zhang J, -W, Wang L, Kang X, -G, Wang J, Duan Q, -L: In Vitro Effects of HAS-2 Gene Silencing on the Proliferation and Apoptosis of the MCF-7 Human Breast Cancer Cell Line. Cell Physiol Biochem 2016; 40:807-817).

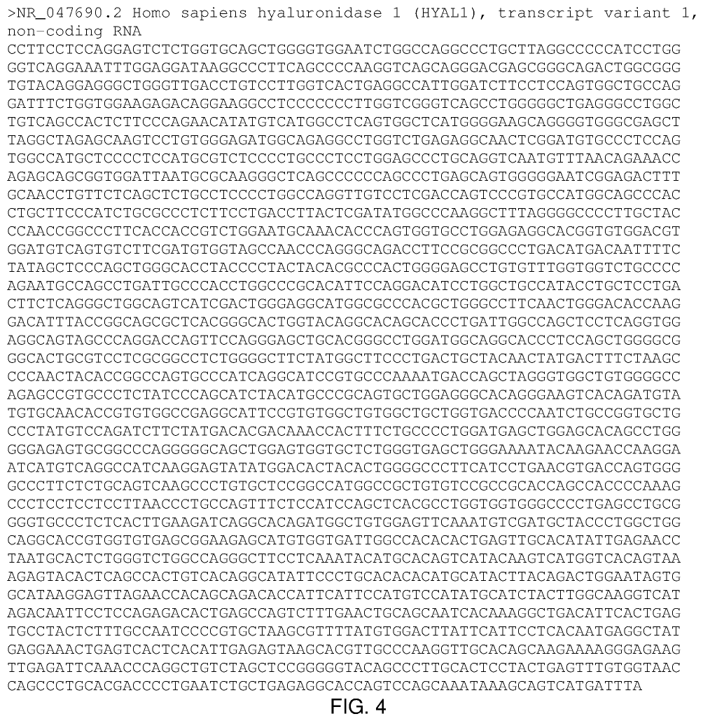

FIG. 4 provides an exemplary mRNA sequence for human HYAL1 (GenBank Accession No. NM_033159.4, e.g., HYAL1 hyaluronidase 1 [Homo sapiens (human)], Gene ID: 3373). HYAL1 also has been knocked down using RNAi agents, including: TTCTCCGAACGTGTCACGT (SEQ ID NO: 17) and GGAAGTCACAGATGTATGT (SEQ ID NO: 18) (See, Tan J X, Wang X Y, Li H Y, Su X L, Wang L, Ran L, Zheng K, Ren G S. HYAL1 overexpression is correlated with the malignant behavior of human breast cancer. Int J Cancer. 2011 Mar. 15; 128(6):1303-15).

As with RNAi therapeutics antisense technology is mature and one of ordinary skill can develop suitable reagents for production of antisense oligonucleotides able to target specific genes, such as HAS2 or HYAL1. (Quemener A M, et al., The powerful world of antisense oligonucleotides: From bench to bedside. Wiley Interdiscip Rev RNA. 2020 September; 11(5):e1594; Antisense LNA® GapmeRs Handbook, Qiagen, October 2017; and Shen X, et al., Chemistry, mechanism and clinical status of antisense oligonucleotides and duplex RNAs. Nucleic Acids Res. 2018; 46(4):1584-1600). Antisense technology has been used to knock down HAS2 (see, e.g., Udabage L, et al., Antisense-mediated suppression of hyaluronan synthase 2 inhibits the tumorigenesis and progression of breast cancer. Cancer Res. 2005 Jul. 15; 65(14):6139-50; and Kolliopoulos C, et al. Has2 natural antisense RNA and Hmga2 promote Has2 expression during TGFβ-induced EMT in breast cancer. Matrix Biol. 2019 July; 80: 29-45) and HYAL1 (see, e.g., Lokeshwar V B, et al., HYAL1 hyaluronidase in prostate cancer: a tumor promoter and suppressor. Cancer Res. 2005 Sep. 1; 65(17):7782-9). Commercial sources of gene-specific development services for antisense reagents include, without limitation Ionis Pharmaceuticals, Inc., among others.

### Example 1

We report a case using UBM, anterior-OCT (Optical coherence tomography) and histological analysis to confirm CCO from a pediatric case of Fanconi anemia (FA). This is a rare (1-5 cases/million), autosomal recessive disorder arising from excess chromosomal breakage, where the characteristics are progressive pancytopenia, elevated risk for tumors (leukemia and solid), and congenital abnormalities—short stature, limb malformations (radial aplasia, thumb aplasia/hypoplasia, or duplicated thumb), skin pigmentation and abnormalities in cardiac, renal, gastrointestinal, endocrine and neuronal development. Ocular manifestations in 48% of cases primarily include short-almond-shaped palpebral fissures, hyper- or hypotelorism, ptosis, microphthalmia, and epicanthal folds, with isolated cases of congenital glaucoma, cataract, uveal or optic disc vasculopathy, retinoblastoma, and corneal clouding. Comparative RNA-Seq analysis of the opaque cornea from our patient with FA to demonstrate gene expression differences in the affected tissue, including metabolic enzymes of glycosaminoglycans, leads us to a novel mechanism in causation of CCO and uncovering of two related players underpinning CCO.

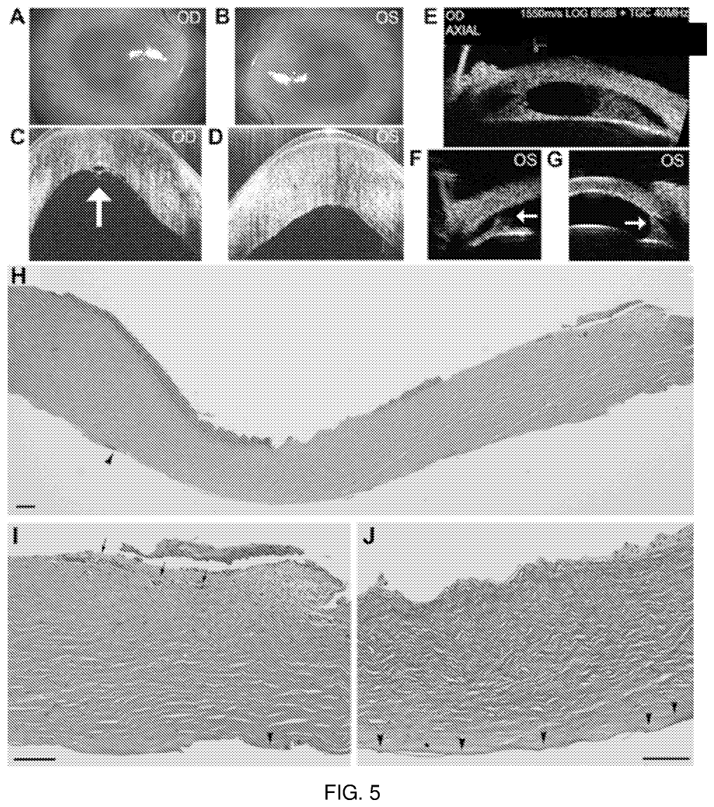

A female pediatric patient diagnosed with FA and poor vision was referred to a pediatric ophthalmologist for evaluation. The patient, at 23 months-old, presented with blurred vision, microphthalmia, peripheral scleralization, microcorneas (FIG. 5 (A)), a visible ectropion uvea, and pendular nystagmus. Upon examination under anesthesia (EUA), she had clear evidence of anterior segment developmental anomalies (ASDA), where the right cornea was more opaque. (FIG. 5 (B)) and displayed a distinct central leukoma. The patient had normal flash visual evoked potentials (VEP). Anterior OCT findings in the right eye showed dehiscence of the Descemet membrane (FIG. 5 (C)) and in the left eye an area of irregularity in the central cornea (FIG. 5 (D)), and hyper-reflective regions observed in both stroma confirmed regions of haze (FIG. 5. (C-D)). UBM revealed iridiocorneal adhesions in both eyes (FIG. 5 (E-G)). Axial lengths were 15 mm OS and 14.5 mm OD. Her raised intracranial pressure was treated with ventriculoperitoneal (VP) shunts. She had a lid lift on her left lid and, thereafter, a left fixation preference. Penetrating keratoplasty (PKP), with 6 mm donor trephine on a 5 mm host cornea, and four peripheral iridotomies were performed on the right eye. The patient started on antibiotic/steroid combination drops eight times daily, with eye ointment at night and cyclopentolate (1%) three times daily. Three months later, her right eye was treated for esotropia by right medial rectus recession (4 mm) and lateral rectus resection (5 mm). She began to prefer right eye for fixation. The last VEP showed same signal amplitude, but slight increase in latency maybe due to her aversion to spectacles. Systemic anomalies included short stature, absent radius, microcephaly, dysplastic kidneys, which are established signs for Fanconi anemia, in addition to bilateral congenital hip dislocation (CHD), a closed atrial-septal defect (ASD) leaving a patent foramen ovale (PFO), and corpus collosum partial agenesis. The ocular family history noted cataracts and nystagmus, and non-ocular noted cancer and rheumatoid arthritis.

The pathology report on the right eye opaque cornea revealed five notable observations: extensive regions lacking Bowman's membrane (FIG. 5 (H)), central corneal thinning, disorganization and vascularization of the corneal stroma (FIG. 5 (I)), irregular and attenuated Descemet's membrane with paucity of corneal endothelial cells (FIG. 5 (I-J)), and keratinization of the corneal epithelium. The first three findings confirmed the relevant OCT observations (FIG. 5 (C-D)).

The patient was diagnosed with Fanconi anemia based on positive DEB (patient: 3.66; normal: 0-0.3) and MMC (patient: 4.26; normal: 0.06-0.24) chromosome breakage tests. The FA gene sequencing panel showed a heterozygous mutation in FANCD2 (c.1278+3_1278+6delAAGT), in addition to single allele mutations of unknown clinical significance in FANCC (c.77C>T (p.S26F) and FANCM (c.4516-5_4516-2delCTTA).

### Materials and Methods

### Clinical Procedures

Case note of patient presenting at UPMC Children's Hospital of Pittsburgh were retrospectively reviewed, including best corrected visual acuity (BCVA), refraction, intraocular pressure (TOP), fundus appearance, and anterior segment phenotype. Slit-lamp biomicroscopy, electrophysiological, and optical coherence tomography (OCT) findings were also recorded.

### Tissue Samples

Control tissues for the study were taken from an age-matched eyeball (The San Diego Eye bank®). The donor eyeball, at 3 yrs 10-month-old, was from a male with history of autosomal dominant polycystic kidney disease (ADPKD) associated with Caroli syndrome. Affected patient (ACo), from which informed consent was obtained, and normal donor (Co) corneal tissues were extracted and collected in accordance with the protocol approved by the University of Pittsburgh Institutional Review Board (PRO13090514).

### RNA Extraction

Cornea from donor eyeball and affected corneal tissue from the patient (extracted during surgery) were immediately submerged in 1.5 ml RNAlater (Thermo Fisher Scientific Inc.) aliquots in 2 ml tubes, placed on ice, and stored at −20° C. Each tissue was cut to small pieces (˜2 mm), washed with PBS and 600 μl buffer RLT with β-mercaptoethanol at 1/10th volume added (Qiagen RNAeasy Mini kit). They were efficiently disrupted using MagNA Lyser Green Beads (prewashed in concentrated nitric acid) and the MagNA lyser instrument (Roche) using 3 cycles of 20 secs disruption and 20 secs on ice. Resulting lysates were centrifuged for 2 mins at full speed, supernatants pipetted into fresh 1.5 ml microfuge tubes, and homogenized using the QIAshredder columns (2 mins spin at 13,000 rpm). Supernatants were loaded onto RNeasy mini spin columns, centrifuged (15 s at 8,000 rpm), columns washed, buffered and then RNase-free water added prior to 1 min centrifugation at 8,000 rpm to elute tissue RNA. The last step was repeated using 30 μl of RNase-free water. Yield and purity of eluents were assessed using the Nano-drop 2000C (Thermo Fisher Scientific Inc).

### RNA Library Preparation, HiSeq Sequencing and Data-Mining

RNA library preparations, sequencing reactions, and data conversion were conducted at GENEWIZ, LLC. (South Plainfield, NJ, USA). RNA libraries were prepared using the NEBNext Ultra RNA Library Prep Kit for Illumina (NEB, Ipswich, MA, USA). Briefly, extracted mRNAs from above corneal preps were enriched by binding to Oligo d(T) beads, fragmented for 5 minutes at 65° C., and first strand and second strand cDNAs synthesized with end-repair and adenylation reactions at 3′ ends. A universal adapter was ligated to cDNA fragments, followed by index addition and library enrichment with limited cycle PCR. A DNA Chip on the Agilent 2100 Bioanalyzer (Agilent Technologies, Palo Alto, CA, USA) was used to validate libraries, and were quantified by the Qubit 2.0 Fluorometer (Invitrogen, Carlsbad, CA) and quantitative PCR (Applied Biosystems, Carlsbad, CA, USA). Sequencing was performed by multiplexing and clustering libraries on one lane of a flow cell, using the Illumina cBOT™ instrument, and loaded into the Illumina HiSeq 2500 sequencer. Samples were sequenced at 1×50 Single Read Rapid Run configuration (depth of 125 million reads per lane). Image analysis and base calling were conducted using the sequencer HiSeq Control Software (HCS). Raw sequence data (.bcl files) was converted into fastq files and de-multiplexed using the Illumina CASSAVA 1.8.2 program. One mismatch was allowed for index sequence identification.

Quality control of RNA-Seq reads were assessed using three criteria: percentage of sample total reads with Phred scores between 30-40 (measure base-calling reliability, where score of 30-40 indicates 99.9-99.99% accuracy of a base call), GC content bias, and 70-90% of reads mapping to the human reference genome (hg38). The CLC Genomics workbench 9.5 program (CLC Bio, Aarhus, Denmark, www.cicbio.com/) was used to map genes to hg38 (genome.ucsc.edu/cgi.bin/hgGateway?db=hg38). Gene expression value for each gene, the TPM (Transcripts Per Kilobase Million), was normalized for total exon-length and total number of matches in an experiment (i.e. uniquely mapped reads to genes are counted and non-unique matches are distributed per ratio to genes). T-tests were performed in log₂transformed data to identify genes with significant differences in expression between phenotypes (p value<0.05, and ≥±1.5-fold differences between phenotypes), and filtered genes were examined by functional analysis using the Ingenuity Pathway Analysis (IPA; Ingenuity Systems, Mountain View, CA; www.ingenuity.com). Comparative transcriptome analysis was performed by comparing our misregulated gene lists to compendium of gene expression studies curated in the correlation engine BaseSpace (Illumina, Inc., San Diego, CA). Common gene expression variation lists between studies were generated by placing misregulated gene lists into a Venn diagram using the web program: Bioinformatics & Evolutionary Genomics (bioinformatics.psb.ugent.be/webtools/Venn/).

### Results

### Corneal Transcriptome of FA Patient Confirms Opaque Profile at Molecular Level and Fibrosis

To determine the molecular underpinnings of CCO in FA, we chose comparative RNA-Seq analysis of our affected patient cornea (ACo) versus an age-matched normal donor cornea (Co). Examination of the Co transcriptome confirmed it represents cornea at the molecular level, as it contains three corneal-enriched keratins in the top 20 highly expressed genes (KRT12, KRT5, and KRT3) and other corneal-specific genes (including the corneal-specific crystallin ALDH3A1, CLU that is highly expressed in the corneal epithelium and the highly-specific corneal stromal stem cell marker NTSE (CD73); Table 1). Twelve genes in the top 20 highly expressed genes are members of the ATP-generating mitochondrial genes, which we suggest are primarily expressed by the corneal endothelium to maintain corneal dehydration and, thus, transparency through ionic pumps. The above findings in the Co transcriptome strongly suggest it represents corneal tissue.

TABLE 1
Top twenty highly expressed genes and highly-specific corneal stromal
gene in the Co transcriptome (age-matched normal cornea).

| Rank | Gene | Description | TPM |
|---|---|---|---|
| 1 | KRT12 | keratin 12 | 42147.27 |
| 2 | MT-ATP6 | mitochondrially encoded ATP synthase membrane subunit 6 | 23380.06 |
| 3 | MT-CO1 | mitochondrially encoded cytochrome c oxidase I | 23272.32 |
| 4 | MT-CO3 | mitochondrially encoded cytochrome c oxidase III | 21317.27 |
| 5 | MT-ATP8 | mitochondrially encoded ATP synthase 8 | 19899.95 |
| 6 | MT-CO2 | mitochondrially encoded cytochrome c oxidase II | 19058.02 |
| 7 | KRT5 | keratin 5 | 17817.47 |
| 8 | MT-ND4 | mitochondrially encoded NADH dehydrogenase 4 | 14518.52 |
| 9 | CLU | clusterin | 9705.36 |
| 10 | FTH1 | ferritin heavy chain 1 | 8364.79 |
| 11 | MT-ND6 | mitochondrially encoded NADH dehydrogenase 6 | 8164.06 |
| 12 | MT-ND3 | mitochondrially encoded NADH dehydrogenase 3 | 8136.48 |
| 13 | MT-CYB | mitochondrially encoded cytochrome b | 7619.32 |
| 14 | MT-ND1 | mitochondrially encoded NADH dehydrogenase 1 | 6327.37 |
| 15 | MT-ND2 | mitochondrially encoded NADH dehydrogenase 2 | 6157.95 |
| 16 | APOD | apolipoprotein D | 5611.91 |
| 17 | MT-ND4L | mitochondrially encoded NADH 4L dehydrogenase | 5380.29 |
| 18 | IFI6 | interferon alpha inducible protein 6 | 5014.16 |
| 19 | KRT3 | keratin 3 | 4929.84 |
| 20 | ALDH3A1 | aldehyde dehydrogenase 3 family member A1 | 4816.33 |
| 8200 | NT5E | 5′-nucleotidase ecto | 14.20 |

Comparative RNA-Seq analysis was conducted to confirm cloudy corneas, at the molecular level in the ACo transcriptome. We focused on corneal stroma-specific genes, as this layer exhibited regions of haze in our patient (FIG. 5 (C-D, H, I)). Out of the 20 highly expressed genes in the normal cornea (Table S1), 3 keratins (KRT12, KRT5, KRT3) were downregulated (Table 2). Both KRT12 and KRT5 lead to fibrosis when downregulated, as do KRT6A and KRT17 when upregulated. Collagens, the next large group of stromal extracellular matrix (ECM) proteins, were observed to be mainly downregulated, but one group was upregulated (COL3A1, COL5A1, COL1A1). This group of collagens are known to lead to fibrosis when overexpressed. LUM (SLRP family member) was found to be significantly upregulated, which consequently also results in a fibrotic response.

TABLE 2
Corneal genes of the stroma misregulated in the ACo transcriptome of the patient.

| Gene | Category | Function in the cornea | | ACo FC (n) |
|---|---|---|---|---|
| KRT12 | keratins | mutations cause Meesmann epithelial corneal | leads to | −5.87 |
| | | dystrophy | fibrosis when | −2.68 |
| KRT5 | | forms cytoskeleton with KRT12 | misregulated | 2.98 |
| KRT6A | | poorly studied | | 8.02 |
| KRT17 | | putative corneal stem cell marker | | |

| | | | |
|---|---|---|---|
| KRT14 | | leads to regeneration of tissue when upregulated | 2.06 |
| KRT3 | | mutations cause Meesmann epithelial corneal dystrophy | −3.52 |
| KRT78 | | poorly studied | −8635.43 |
| KRT80 | | poorly studied | −2589.38 |
| COL4A4 | collagens | mutations in COL4A5 (X-linked), or COL4A3 and COL4A4 | −13.80 |
| COL4A3 | | (autosomal recessive) result in basement membrane disruption and | −5.29 |
| COL4A5 | | are associated with corneal opacities. | −2.97 |
| COL12A1 | | mutations lead to collagen VI-like syndromes, which generally | −6.32 |
| COL6A2 | | leads to extreme corneal thinning and fragility. | −6.03 |
| COL6A1 | | | −2.89 |
| COL3A1 | | upregulation of these genes results in immune responses leading to | 5.33 |
| COL5A1 | | fibrosis in many organs and tissues, such as kidney, liver, lungs | 2.35 |
| COL1A1 | | and skin. Col5 is a minor collagen that usually intercalates with | 2.28 |
| | | Col1, a major collagen. | |
| LAMA5 | laminin | mutation causes complex ECM syndrome. | −14.61 |
| LUM | SLRP | member of class II SLRPs (small leucine-rich proteoglycans), | 3.67 |
| | | made of keratan sulfate proteoglycans, and recognized as major | |
| | | component of the corneal stroma. It contributes to correct | |
| | | formation of collagen fibrils, and promotes cell adhesion and | |
| | | migration. In tissue fibrosis (e.g. lung, colon), lumican | |
| | | upregulation promotes fibrocyte differentiation. | |

Abbr: ACo, affected cornea; FC, fold-change; n, number; SLRP, small leucine-rich proteoglycan.

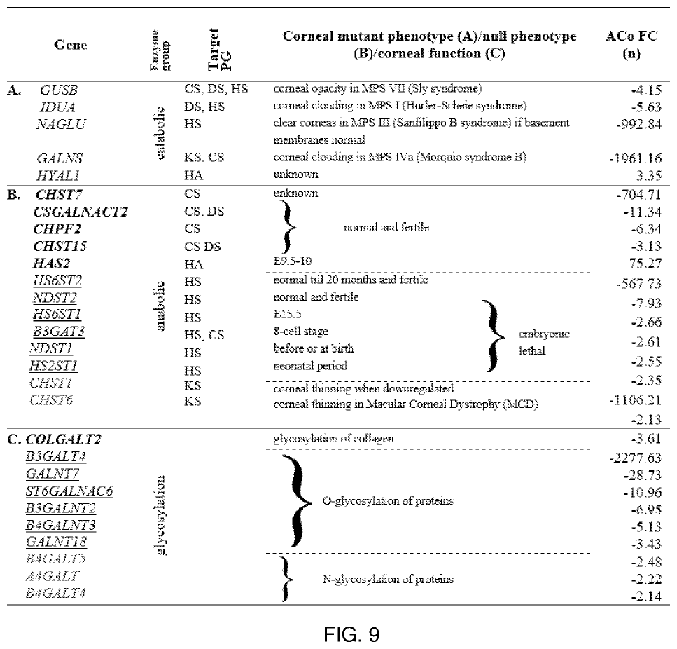

We next analyzed expression variation in enzymes associated with either glycosaminoglycan (GAG) metabolism or glycosylation (Table 3, FIG. 9), particularly downregulated expression as their effects are known. We found all GAG chains (chondroitin sulfate (CS), dermatan sulfate (DS), keratan sulfate (KS), heparan sulfate (HS)) are affected, which reportedly leads to corneal clouding/opacities (GUSB, IDUA, GALNS). For anabolic enzymes, CHST1 and CHST6 (KS chains) were significantly downregulated which both contribute to corneal thinning when their expression is reduced. Analysis of upregulated GAG metabolic enzymes associated with HA, only HYAL1 (3.35) and HAS2 (75.27) were significantly upregulated. The former generates pro-inflammatory hyaluronan fragments, whilst the latter partakes in fibrosis of various tissues when upregulated. We found COLGALT2 as the only glycosylation enzyme concerned with collagen glycosylation that was significantly affected. With glycoprotein glycosylation in the corneal stroma, we found twice as many o-glycosylation enzymes at serine/threonine bases (B3GALT4, GALNT7, ST6GALNAC6, B3GAL NT2, B4GALNT3, GALNT18) are downregulated than those involved in n-glycosylation at asparagine bases (B4GALT5, A4GALT, B4GALT4). Both lead to diminished lubrication of the corneal surface and subsequent epithelial keratinization.

To summarize, results from expression variation in the ACo transcriptome confirm an opaque cornea due to downregulation of corneal-specific genes that lead to opacities (KRT12, KRT3, COL4A4, COL4A5, COL4A3, GUSB, IDUA, GALNS), corneal thinning (COL12A1, COL6A2, COL6A1, CHST1, CHST6) and keratinization of the corneal epithelium (B3GALT4, GALNT7, ST6GALNAC6, B3GALNT2, B4GALNT3, GALNT18, B4GALT5, A4GALT, B4GALT4). In addition, the ACo transcriptome shows an abundance of fibrotic/inflammatory responses (downregulation of KRT12, KRT5, KRT6A, KRT17, CHI3L1, CLU and upregulation of COL3A1, COL5A1, COL1A1, LUM, HAS2).

### Comparison of Patient Corneal Transcriptome Variation to Those From Anemia-Related Studies Uncovers two Important Genes in Fibrosis and Wound-Healing

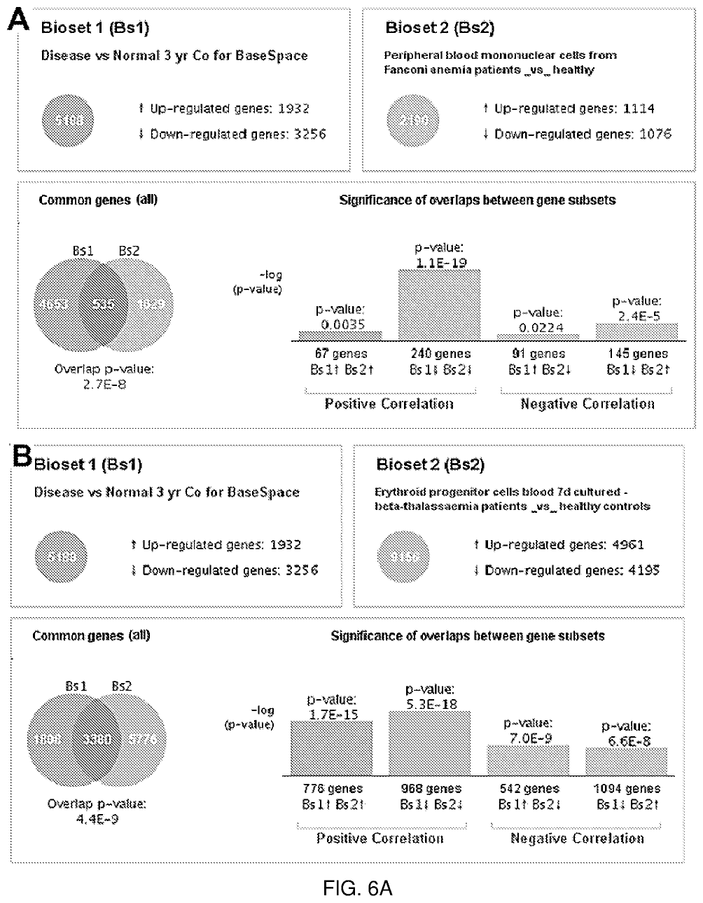

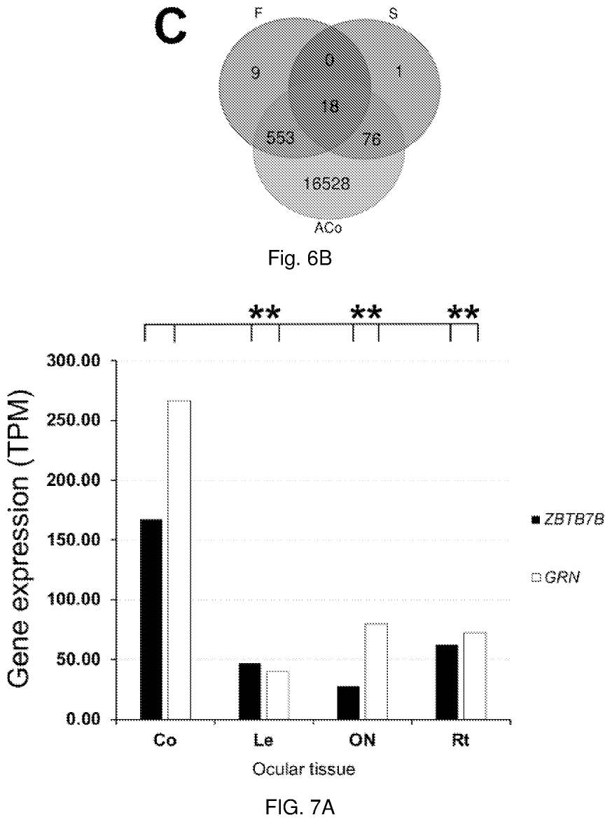

To identify the cause of the corneal fibrosis event in our patient corneas, we employed comparative transcriptome variation analysis (COTVA). This technique has been greatly facilitated by the creation of the Illumina BaseSpaceR correlation engine, which permits large-scale pairwise comparisons of variation in a study transcriptome to those from 22,562 other gene expression studies. Using this correlation engine, ACo transcriptome variation compared favorably with those from two anemia-related studies (FIG. 6). The first study (GSE17233), analyzing peripheral blood transcriptome variation between one normal individual and two FA patients with undescribed ocular anomalies, had 67 common upregulated genes (p-value=0.0035) and 240 downregulated genes in common (pvalue=1.1e-19) (FIG. 6A (A)). The other study (GSE56088), analyzing erythroid progenitor cell transcriptomes cultured for 7 days between beta-thalassaemia patients and healthy controls, had 776 common upregulated genes (p-value=1.7e-15) and 968 downregulated genes (p-value=5.3e-18) (FIG. 6A (B)). Common misregulated genes amongst these 3 studies were identified by placing each study misregulated genes into a Venn diagram format (FIG. 6B (C)). Analysis of the resulting eighteen common genes (Table 4) uncovered the top common downregulated gene as ZBTB7B, which is a zinc finger transcription factor that prevents fibrosis by repressing type I collagen genes-established marker of fibrosis. GRN, a growth factor and potent regulator of wound-healing, is another key common downregulated gene.

TABLE 4
Common 18 genes between the ACo, PBMFA (peripheral blood mononuclear
cells from Fanconi anemia patients) and EPCBT (erythroid progenitor
cells from beta-thalassaemia patients) studies.

| | | | RNA-seq fold changes (n) | | |
|---|---|---|---|---|---|
| # | Gene | Description | ACo | PBMFA | EPCBT |
| 1 | ZBTB7B | zinc finger and BTB domain containing 7B | −7.107 | −11.2 | −2.26 |
| 2 | EIF4G1 | eukaryotic translation initiation factor 4 gamma 1 | −7.009 | −2.95 | −2.99 |
| 3 | TAP1 | transporter 1, ATP binding cassette subfamily B | −6.061 | −2.36 | −2.05 |
| 4 | RELA | RELA proto-oncogene, NF-kB subunit | −3.337 | −2.07 | −4.22 |
| 5 | AQP1 | aquaporin 1. Dominant in Co endothelium | −3.212 | −2.58 | −2.52 |
| 6 | HK1 | hexokinase 1 | −3.125 | −2.79 | −2.16 |
| 7 | GRN | granulin precursor | −2.75 | −2.19 | −3.53 |
| 8 | ASCC2 | activating signal cointegrator 1 complex subunit 2 | −2.582 | −2.93 | −3.93 |
| 9 | PPP4R1 | protein phosphatase 4 regulatory subunit 1 | −2.299 | −2.83 | −2.64 |
| 10 | RAD23A | RAD23 homolog A, nucleotide excision repair | −2.286 | −2.07 | −6.56 |
| 11 | HLA-B | major histocompatibility complex, class I, B | −2.055 | −34.1 | −16 |
| 12 | HLA-A | major histocompatibility complex, class I, A | −2.013 | −28.4 | −13.7 |
| 13 | STX10 | syntaxin 10 | −2.011 | −2.67 | −2.08 |
| 14 | TMEM205 | transmembrane protein 205 | 2.352 | 2.19 | 2.96 |
| 15 | SCOC | short coiled-coil protein. | 2.537 | 2.23 | 2.47 |
| 16 | EIF3M | eukaryotic translation initiation factor 3 subunit M | 2.601 | 5.38 | 5.18 |
| 17 | COQ5 | coenzyme Q5, methyltransferase | 2.626 | 7.37 | 2.05 |
| 18 | EEF1B2 | eukaryotic translation elongation factor 1 beta 2 | 3.646 | 2.95 | 4.06 |

### Confirmation of ZBTB7B as Anti Fibrotic and GRN as Wound-Healing Agents in the Cornea

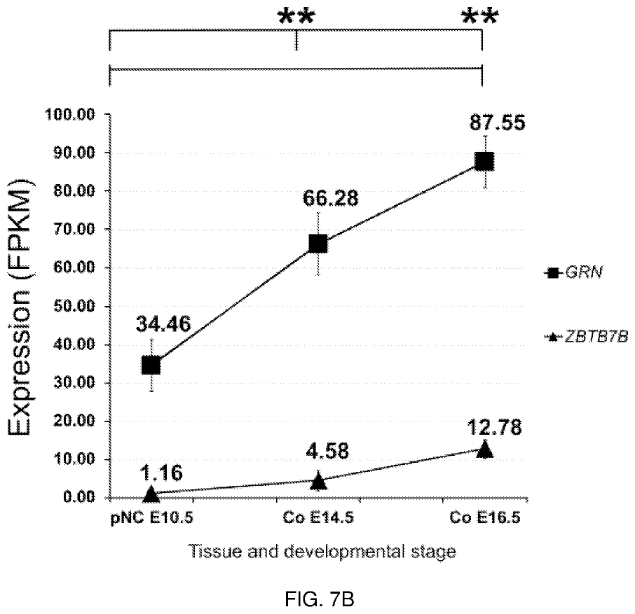

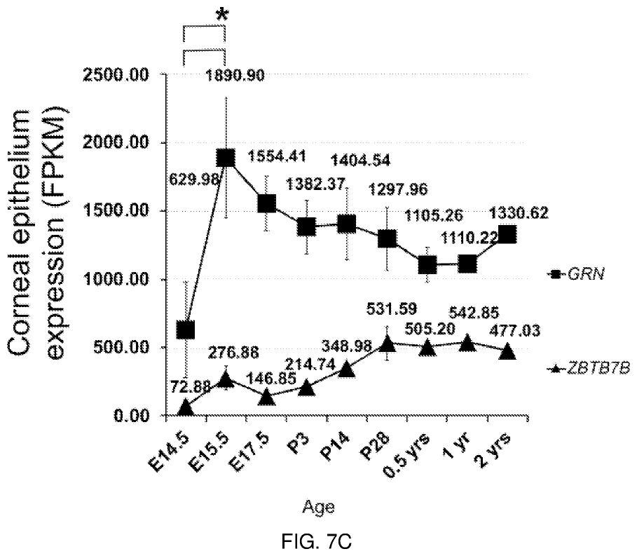

We looked at expression of both genes amongst four ocular tissues, specifically the cornea, lens, optic nerve and retina, and both were found to be significantly highly expressed in the cornea relative to the other tissues (FIG. 7A). To analyze their expression during cornea development, we searched publicly available gene expression studies in the NCBI Gene Expression Omnibus (GEO, http://www.ncbi.nlm.nih.gov/geo/). Two microarray analyses (GSE121044 and GSE43155) were identified that follow gene expression across embryonic and postnatal stages of mouse corneal development. We found both genes increased expression during the prenatal stages (from GSE121044; FIG. 7B), where both peaked at ˜E15.5 (from GSE43155; FIG. 7C) but ZBTB7B also increased steadily during the postnatal period.

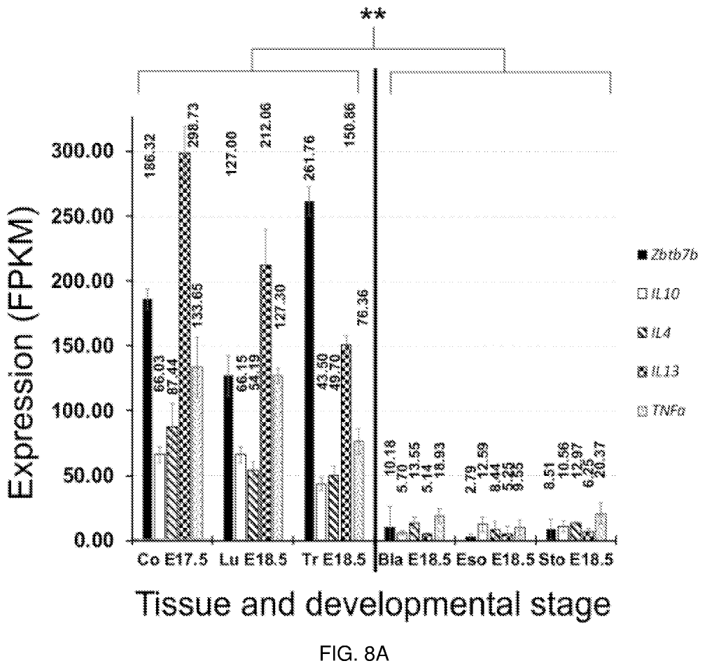

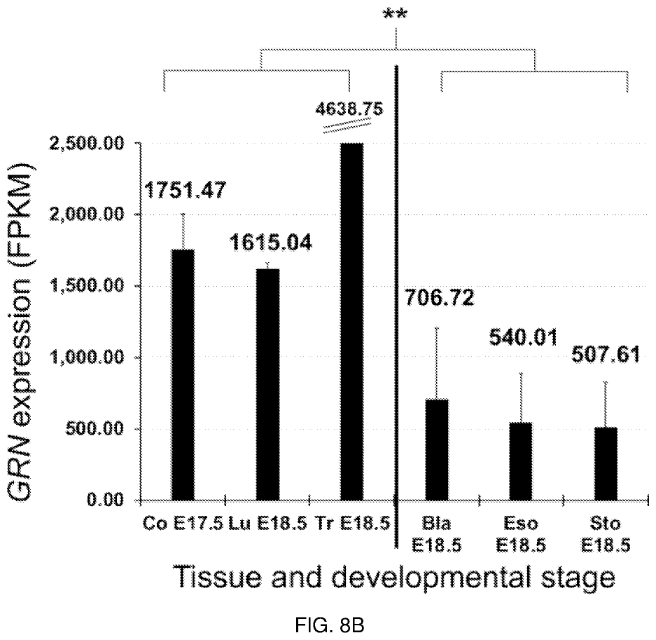

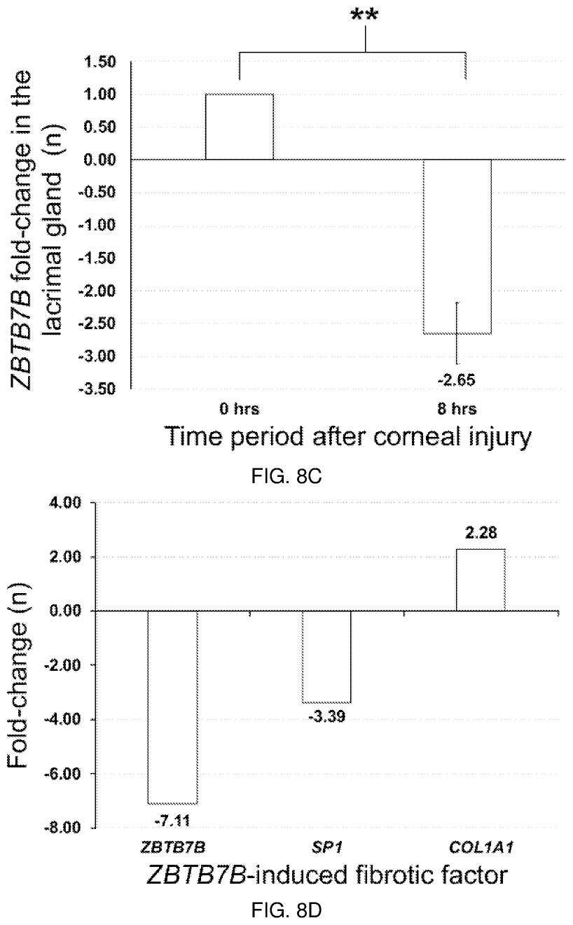

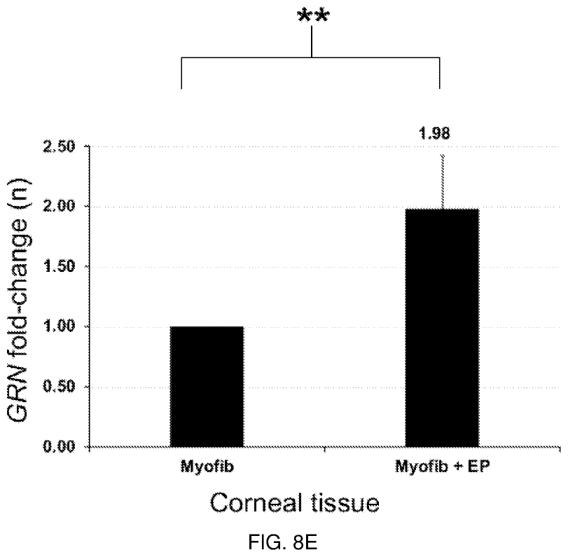

A search in NCBI GEO for gene expression studies of the corneal epithelia in connection to fibrosis and wound-healing revealed 2 relevant studies. Dataset GSE43381 identifies genes enriched in mouse cornea, bladder, esophagus, lung, proximal small intestine, skin, stomach, and trachea epithelia. We found, unexpectedly, high expression of ZBTB7B and GRN in the cornea, lung and trachea epithelia relative to those found in the bladder, esophagus, and stomach epithelia (FIG. 8 (A, B)). Expression of several important anti-inflammatory players that prevent fibrosis, IL4, IL10, IL13, and TNFa, were also found to follow the same pattern (FIG. 8 (A)). These results suggest ZBTB7B and GRN are highly expressed in those tissues where anti-inflammatory responses are significant.

This above finding led to two hypotheses, where the first was that downregulation of the antifibrotic factor ZBTB7B in injured corneas leads to fibrosis in the tissue that subsequently leads to opacities. Confirmation of this theory comes from the microarray-based study on chemical burns (silver nitrate application) to mice cornea that results in representative gene expression changes in the lacrimal gland. Both corneas became opaque, and corresponding gene expression changes in the lacrimal gland reflective of expression variation in the cornea were examined post-injury compared to non-injured mice. We uncovered significant downregulation in ZBTB7B (−2.65) in the lacrimal gland 8 hrs post-injury (FIG. 8 (C)), thus, confirming our hypothesis that ZBTB7B downregulation in corneal injury may contribute to opacities in the tissue. A study comparing scleroderma (fibrotic skin) fibroblasts to normal ones where the fibrotic marker COL1A1 was shown to be upregulated upon ZBTB7B and Sp1 downregulation further confirms our hypothesis, because the ACo transcriptome also shows downregulation of ZBTB7B, Sp1 and upregulation of COL1A1 (FIG. 8 (D)).

In the second hypothesis, we propose GRN is important for wound-healing in damaged corneas. A study showing this comes from analysis of corneal wound-healing after ethyl pyruvate addition to culture medium where prior TGFb1 treatment leads to corneal keratocytes forming myofibroblasts. Subsequent ethyl pyruvate addition leads to modifying the TGFb1-driven transition of keratocytes to myofibroblasts by inhibiting upregulation of profibrotic genes, thus replicating the wound-healing response. We identify a significant increase in GRN expression in the dataset after ethyl pyruvate addition (FIG. 8E), thus confirming GRN in the cornea contributes to wound-healing.

In sum, this is the first report of RNA-Seq gene expression analysis on an opaque cornea from a patient with FA. Clinical examination revealed bilateral microcornea in the patient (FIG. 5 (A)), which has been reported in 55-100% of FA seen in the clinic whereas CCO has not been previously described in FA. External exam and anterior-OCT established corneal haze in both eyes with increased opacity in the right cornea (FIG. 5 (B-D)). Histopathology analysis confirmed the anterior-OCT findings (FIG. 5 (C,D)) that the haze was in the corneal stroma (FIG. 5 (I)), in addition to uncovering corneal membranes were disrupted (FIG. 5 (I-J)), central cornea was irregular, and the epithelium was keratinized.

Findings of opacity in the corneal stroma directed our subsequent RNA-Seq analysis towards proteins highly expressed there (Table 2), which was determined from those that were found to be highly expressed in a normal cornea (Table 1). Certain keratins (KRT12, KRT5, KRT3) were downregulated and some upregulated (KRT6A and KRT17), as were specific collagens (COL3A1, COL5A1, COL1A1), which previous studies demonstrated lead to fibrosis. The SLRP family member LUM, proven to result in fibrosis when upregulated, was also over-expressed. These findings suggested the patient cornea has undergone opacity-generating fibrosis, which has been proven in the cornea when infected, injured or post-surgery.

Features of the patient corneal histopathology were confirmed in the transcriptome when we analyzed expression variation in GAG metabolism or glycosylation enzymes (Table 3). This approach was taken as major fibrous proteins and proteoglycans in the corneal stroma are either heavily dependent on glycosylation or contain GAGs respectively. We found catabolic enzymes targeted all GAG chains and produce corneal haze when downregulated (GUSB, IDUA, GALNS). NAGLU is the only exception, which has exhibited opaque corneas in MPS III only upon massive HS accumulation where basement membrane disruption was also evident. Interesting anabolic enzymes were those targeting KS chains (CHST1 and CHST6), which cause corneal thinning when downregulated. Nine members of o- and n-glycosylation enzymes were downregulated (B3GALT4, GALNT7, ST6GALNAC6, B3GALNT2, B4GALNT3, GALNT18), which results in epithelial keratinization from decreased corneal surface lubrication. Upregulated GAG metabolic enzymes were associated with hyaluronan metabolism, HYAL1 and HAS2; the first generates pro-inflammatory hyaluronan fragments and the second partakes in tissue fibrosis when upregulated, thus adding further evidence on opacity-causing fibrosis in the patient cornea.

To determine molecular players of fibrosis in the patient cornea, we conducted comparative transcriptome analysis using the Illumina BaseSpaceR correlation engine. Two anemia-related studies had a significant correlation with the affected cornea transcriptome (FIG. 6A (A-B)), which were considered reasonable as blood and parts of the cornea (i.e. stroma) are derived from embryonic mesoderm. For the same reason, fibrotic genes could be identified from these comparisons without corneal opacities being prevalent in FA or anemia-related disease. When common misregulated genes between these datasets were placed in a Venn diagram (FIG. 6B (C)), ZBTB7B and GRN were identified (Table 4) that play roles as anti-fibrotic and wound repair factors, respectively.

We showed that ZBTB7B and GRN are highly expressed, together with anti-inflammatory markers (IL4, IL10, IL13 and TNFa), in tissue epithelia where fibrosis would be considered fatal (lungs and trachea) in contrast to epithelia where fibrosis is not fatal (bladder and stomach) (FIGS. 8A and 8B). We also show that they are both highly expressed in the cornea (FIG. 7A), therefore indicating their importance in preventing fibrosis and, therefore, blindness by opacity formation. In demonstrating ZBTB7B has a role in preventing fibrosis and corneal opacities, we focused on a study in which rabbit eyes were exposed to chemical burns through silver nitrate application and reflective gene expression changes in the cornea were monitored by gene expression analysis of the lacrimal glands. Gene expression changes in the lacrimal glands reflective of those in the cornea has long been proven. Our processing of the raw data from lacrimal gene expression changes to show reduction of ZBTB7B in this model of chemical burns-inducing corneal opacities (FIGS. 8C and 8D) is thus evident that decrease in the factor may play a role in CCO causation. For GRN role in wound-healing, we choose a TGFb1-induced fibrosis study where conversion of keratocytes to myofibroblasts was reversed by ethyl pyruvate addition (S. A. Harvey, S. A., et al., Responses of cultured human keratocytes and myofibroblasts to ethyl pyruvate: a microarray analysis of gene expression Invest Ophthalmol Vis Sci 51 (2010) 2917-2927). We uncovered that GRN is increased (FIG. 8E) in the wound-healing type reversal, therefore suggesting it could be involved in wound-healing of the cornea.

Expanding the number of CCO cases to demonstrate basement membrane disruption and analyze gene expression through RNA-Seq technologies to establish opacity-causing fibrosis would be a necessary step forward in confirming our hypothesis for CCO causation. Studies of mice where ZBTB7B/GRN would be specifically reduced in the developing cornea to induce opacity-causing fibrosis and rescuing the ensuing corneal opacity through over-expression of GRNIZBTB7B would prove the roles of these factors in corneal opacity.

### Example 2

ZBTB7B is a transcription factor that we may overexpress in the cornea to prevent corneal scarring in the case of injury, infection, surgery, or congenital disease. Subconjunctival gene delivery of the transcription factor as a non-viral polyplex (successfully shown for another transcription factor that slowed down corneal neovascularization (Yoon et al., 2009) or through rAAV-based gene therapy (Alvarez-Rivera et al., 2020; Bastola et al., 2020; Miyadera et al., 2020) may be used for therapeutic purposes.

GRN has recently been found to be a potent wound-healing factor as exemplified by human parasitic liver fluke Opisthorchis viverrini granulin family member Ov-GRN-1. The human precursor protein progranulin, PGRN, has seven modules that each have differing functions. We can explore structure-wound-healing activity of these modules GRN1-GRN7, and use (or manipulate) one or combination of these in identifying the most effective in wound-healing of corneal scars to achieve transparency. Our data suggests that GRN4 (e.g., amino acids 281-226 of SEQ ID NO: 11), GRN6 (e.g., amino acids 442-˜496 of SEQ ID NO: 11), and GRN7 (e.g., amino acids ˜518-573 of SEQ ID NO: 11) are the most down-regulated in the FA CCO cornea. Once the most effective protein product of GRN is verified, we can grow this protein to administer to opaque corneas resulting from case injury, infection, surgery or congenital disease.

For opaque corneas showing HAS2/HYAL1 overexpression, we can use HA-blocking peptides that have been previously shown to dampen the inflammatory and fibrotic responses of HA fragments resulting from over activity of HAS2/HYAL1 (Collins et al., 2011).

Our data suggests that ZBTB7B is downregulated in our FA CCO cornea. Isoforms 1 (e.g., NP_001243384.1), 7, 6, 3, and 4 (in order of decreasing down-regulation) were the most significantly down-regulated.

Upstream regulators of HAS2 and HYAL1 are numerous and will be explored to potentially down-regulate target gene expression.

ZBTB7B therapeutics may be delivered intraocularly (subconjunctival or intrastromal injection) in addition to contact lens wear, whereas GRN can be delivered topically. Therapeutics for CCO showing HAS2/HYAL1 overexpression can be delivered also through the topical route.

Having described this invention, it will be understood to those of ordinary skill in the art that the same can be performed within a wide and equivalent range of conditions, formulations and other parameters without affecting the scope of the invention or any embodiment thereof.

1. A method of treating corneal fibrosis in a patient, comprising, reducing expression of HYAL1 and/or HAS2 in the eye of the patient.

2. The method of claim 1, comprising knocking down expression of HYAL1 and/or HAS2 in the eye of the patient. RNAi agent

3. The method of claim 2, comprising administering an RNAi agent and/or an antisense agent to the eye of the patient in an amount effective to treat corneal opacity in the patient.

4. (canceled)

5. The method of claim 3, wherein the RNAi agent is administered to the patient's eye by administration to the patient's eye of a nucleic acid comprising a gene for expressing the RNAi agent.

6. (canceled)

7. The method of claim 1, comprising knocking down HYAL1 in the eye of the patient.

8. The method of claim 1, comprising knocking down HAS2 in the eye of the patient.

9. (canceled)

10. A method of treating corneal fibrosis in a patient, comprising, administering a granulin polypeptide and/or a ZBTB7B polypeptide to a patient's eye, in an amount effective to reduce corneal fibrosis in the patient.

11. The method of claim 10, comprising administering a granulin polypeptide to a patient's eye, in an amount effective to reduce corneal opacity, corneal fibrosis, or corneal scarring in the patient.

12. The method of claim 11, wherein the granulin polypeptide comprises or consists of:
the amino acid sequence of SEQ ID NO: 11,
an amino acid sequence having at least 70, 80, 90, 95, 98, or 99% sequence identity or similarity to SEQ ID NO: 11, or
an amino acid sequence comprising one or more granulin motifs, such as X₂₋₃CX₅₋₆CX₅CCX₈CCX₆CCX₅CCX₄CX₅₋₆CX₂ or X₃CX₆CX₅CCX₇CC, where each instance of X is, independently, any amino acid, or the granulin polypeptide has the amino acid sequence of any one of SEQ ID NOS: 1-10 and 19-24, or a sequence having at least 70, 80, 90, 95, or 98% sequence identity or similarity to any one of SEQ ID NOS: 1-10, 19, or 20.

13. (canceled)

14. The method of claim 11, wherein the granulin polypeptide is a granulin 4, granulin 6, or granulin 7 polypeptide, or a combination of any of the preceding.

15. The method of claim 11, wherein a nucleic acid is administered to the patient's eye, comprising a gene for expressing the granulin polypeptide in the patient's eye, to produce the granulin polypeptide in the patient's eye or an mRNA encoding the granulin polypeptide is administered to the patient's eye to produce the granulin polypeptide in the patient's eye.

16-18. (canceled)

19. The method of claim 10, comprising administering a ZBTB7B polypeptide to a patient's eye, in an amount effective to reduce corneal opacity, corneal fibrosis, or corneal scarring in the patient.

20. The method of claim 19, wherein the ZBTB7B polypeptide has the amino acid sequence of SEQ ID NO: 13, or a sequence having at least 70, 80, 90, 95, 98, or 99% sequence identity or similarity to SEQ ID NO: 13.

21. The method of claim 19, wherein a nucleic acid is administered to the patient's eye, comprising a gene for expressing the ZBTB7B polypeptide in the patient's eye, to produce the ZBTB7B polypeptide in the patient's eye or an mRNA encoding the ZBTB7B polypeptide is administered to the patient's eye to produce the ZBTB7B polypeptide in the patient's eye.

22-25. (canceled)

26. The method of claim 10, comprising administering both of a granulin polypeptide and/or a ZBTB7B polypeptide to the patient's eye.

27. The method of claim 10, comprising both administering a granulin polypeptide and/or a ZBTB7B polypeptide to the patient's eye or cornea, and knocking down expression of HYAL1 and/or HAS2 in the patient's eye or in the patient's cornea.

28. The method of claim 1, wherein the patient has a congenital corneal opacity, an acquired corneal opacity, corneal trauma, or a corneal transplant.

29-31. (canceled)

32. The method of claim 1, wherein the patient has Fanconi anemia, Peters' anomaly (PA), sclerocornea, congenital hereditary endothelial dystrophy (CHED), congenital hereditary stromal dystrophy (CHSD), posterior polymorphous dystrophy (PPMD), congenital anterior staphyloma, granular corneal dystrophy, cystinosis, ichthyosis, trisomy 8 mosaicism, or Farber's disease.

33. The method of claim 1, wherein the patient is a human.

34. (canceled)

35. A topical or parenteral pharmaceutical composition for delivery to the eye of a patient, comprising a granulin polypeptide and/or a ZBTB7B polypeptide, a nucleic acid encoding a granulin polypeptide and/or a nucleic acid encoding ZBTB7B polypeptide, an antisense or RNAi agent for knocking down expression of HYAL1 and/or HAS2 in the patient's eye or in the patient's cornea, and/or a nucleic acid encoding RNAi agent for knocking down expression of HYAL1 and/or HAS2 in the patient's eye or in the patient's cornea, and a pharmaceutically acceptable excipient or carrier.


SEQUENCE LISTING (26 sequences)

SEQ ID NO: 1 - protein, 57 aa, Homo sapiens
GGPCQVDAHCSAGHSCIFTVSGTSSCCPFPEAVACGDGHHCCPRGFHCSADGRSCFQ

SEQ ID NO: 2 - protein, 58 aa, Homo sapiens
AIQCPDSQFECPDFSTCCVMVDGSWGCCPMPQASCCEDRVHCCPHGAFCDLVHTRCIT

SEQ ID NO: 3 - protein, 57 aa, Homo sapiens
VMCPDARSRCPDGSTCCELPSGKYGCCPMPNATCCSDHLHCCPQDTVCDLIQSKCLS

SEQ ID NO: 4 - protein, 54 aa, Homo sapiens
CDMEVSCPDGYTCCRLQSGAWGCCPFTQAVCCEDHIHCCPAGFTCDTQKGTCEQ

SEQ ID NO: 5 - protein, 56 aa, Homo sapiens
DVKCDMEVSCPDGYTCCRLQSGAWGCCPFTQAVCCEDHIHCCPAGFTCDTQKGTCE

SEQ ID NO: 6 - protein, 56 aa, Homo sapiens
DVPCDNVSSCPSSDTCCQLTSGEWGCCPIPEAVCCSDHQHCCPQGYTCVAEGQCQR

SEQ ID NO: 7 - protein, 57 aa, Homo sapiens
DIGCDQHTSCPVGQTCCPSLGGSWACCQLPHAVCCEDRQHCCPAGYTCNVKARSCEK

SEQ ID NO: 8 - protein, 55 aa, Homo sapiens
IGCDQHTSCPVGQTCCPSLGGSWACCQLPHAVCCEDRQHCCPAGYTCNVKARSCE

SEQ ID NO: 9 - protein, 56 aa, Homo sapiens
DVECGEGHFCHDNQTCCRDNRQGWACCPYRQGVCCADRRHCCPAGFRCAARGTKCL

SEQ ID NO: 10 - protein, 30 aa, Homo sapiens
TRCPDGQFCPVACCLDPGGASYSCCRPLLD

SEQ ID NO: 11 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 12 - DNA, 2130 bp, Homo sapiens
gctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcttaa

SEQ ID NO: 13 - protein, 539 aa, Homo sapiens
MGSPEDDLIGIPFPDHSSELLSCLNEQRQLGHLCDLTIRTQGLEYRTHRAVLAACSHYFKKLFTEGGGGAVMGAGGSGTATGGAGAGVCELDFVGPEALGALLEFAYTATLTTSSANMPAVLQAARLLEIPCVIAACMEILQGSGLEAPSPDEDDCERARQYLEAFATATASGVPNGEDSPPQVPLPPPPPPPPRPVARRSRKPRKAFLQTKGARANHLVPEVPTVPAHPLTYEEEEVAGRVGSSGGSGPGDSYSPPTGTASPPEGPQSYEPYEGEEEEEELVYPPAYGLAQGGGPPLSPEELGSDEDAIDPDLMAYLSSLHQDNLAPGLDSQDKLVRKRRSQMPQECPVCHKIIHGAGKLPRHMRTHTGEKPFACEVCGVRFTRNDKLKIHMRKHTGERPYSCPHCPARFLHSYDLKNHMHLHTGDRPYECHLCHKAFAKEDHLQRHLKGQNCLEVRTRRRRKDDAPPHYPPPSTAAASPAGLDLSNGHLDTFRLSLARFWEQSAPTGPPVSTPGPPDDDEEEGAPTTPQAEGAMESS

SEQ ID NO: 14 - DNA, 3603 bp, Homo sapiens
ggaaagcaagctggaggacaggtgagacagcaggacaggaccggagcagggccccaagcccccgggcctggtgggggacgcgcttcttcccacactgtgagcctcagcagctccagccagcggacccgacggctgagaggagaagatggggagccccgaggatgacctgattgggattccattcccggaccacagcagtgagctcctgagctgcctcaatgagcagcgccagctgggccacctatgtgacctcaccatccggacgcagggccttgaataccgcacccacagggctgtgctagctgcctgtagccactacttcaagaagcttttcactgagggcggtggcggagctgtcatgggggccgggggtagcgggacggccactgggggagcaggggccggtgtgtgtgagctggactttgtagggccagaggcactaggcgccctccttgaatttgcctatacagccacactgaccaccagcagcgccaacatgccagctgtgctccaggctgcccgcctgctggagatcccgtgtgtcatcgctgcttgcatggagattctgcagggcagtgggctagaagctcccagcccggacgaggatgactgtgagcgagcccgccagtatctggaggcctttgccacagccacggcctctggagttcccaatggtgaagacagtcctccacaggtgcccctcccaccacctccgccaccgccacctcggcctgttgcccgccgcagccgcaagccccggaaagctttcctgcaaaccaagggggccagagcaaaccacctagtccctgaggtgcccacagtgcccgcccatcccttgacctatgaggaggaggaggtggcgggcagagtgggcagcagtgggggcagtgggccgggggacagctacagccctcccacaggaactgcctcccctcctgagggtccccagagctacgaaccctatgagggtgaggaagaagaagaggagctggtatatcccccagcctatgggctggcgcagggtggcgggcccccgctgtccccagaggagctgggctcagatgaggatgccatcgatcctgacctgatggcctacctaagctccctgcaccaggacaacctggcaccaggcctggacagccaagacaagctggtgcgcaaacgccgctcccagatgcctcaggagtgccctgtctgccacaagatcatccatggggcaggcaaactgcctcgccacatgaggacccacacaggcgagaagccctttgcctgcgaggtctgcggtgttcgattcaccaggaacgacaagctgaagatccacatgcggaagcacacgggagagcgcccctactcatgcccgcactgcccagcccgcttcctgcacagctacgacctcaagaaccacatgcacctgcacacaggggaccggccctatgagtgccacctgtgccacaaggctttcgccaaggaggaccacctgcagcgccacctcaaaggccagaactgcctggaggtgcgcacccgacggcgccgcaaggacgatgcaccaccccactacccaccaccctctaccgctgctgcatcccccgctggcctcgacctctccaatggccacctggacaccttccgcctctctctagctcgattctgggagcagtcagcccccactgggcccccggtctctaccccagggccccctgatgacgatgaggaggaaggggcacccaccacaccccaggctgaaggtgccatggagtcctcttaaagagggacgagggccagactgaagcagcacaaggccggggacacccatgccaagcagtgggagcacgcaggacagacacagcaggggtctggggcacggagccttgctggcatcagcatcagcccttcctcccagagccctcattccaattccaagctaagaaggtattggggcagaggctccccaaattggggtgatcccccaaggagtgatacatatattgtgtatatatttacagctgtattgtaaaagtggggtccctgtccccagctgctcctggggagtagaagcaataatgtatttctaatttgtgggtcccacttcggctatgcgggtttctagggggtgggggcttgggaccaaagccttgccccgcccctatgccccttgggggttttggctgtgtaagggggtgaaggactgcccctccctttcgagacccctccttcctggtttctgttcctttttcctggcagtgaattatgcaaagggggccggcaaaggaagggtaggtgggggaaagccaggtggaagcttgaaagactgggggactgggcctgtaaggaaggagccatcccagtccccctccgccctgctcccggcgctgagtcatggggtcgtggagaagggggcggggtggcctgattggctcgcctgcccctgggggcagtagaggggccccgcccagctaggggagccgctccgttccactcccctccctagccctccctccccacggccctgggcagggaatgtcttgttcccgccgctccctccccggggccagagggcagggcgggccgggcggcgtcctaccctcttctcctcctccccatctcctccccgcccaggtgcgagccggagccgccgccaccgctgccgcccctgactcacgccgcccccgggctggcgcagcgaagggtgtgggacagggtaaggggttggaagagccttgtggagagcgggcgagccggcgccatctggcggccatgctctgagtgggcgagcgccccccgcggccactggagcgagctgtcttcacgctcctcatccaccccagctggtgagcggcgcccccttgccaaggcagtgggcacagaacttctcgcttggccgcaggggaaggggctgcggacctgtgggaaagtgatccccttcccagatccttgccagccgggcttcctgtcaggcaggggagaataatccccactctgctcttaggattgaatccacccccattctgtacatagcctcttctgttggtcttgttgaaatctagtttcagatttttaactacccaattctgctgggggtgggggacacccccccttcctcgctgggtgctggaccccttttgcagcctgggctctgccttgcactatttccccttcctggcctgacggctcctccccctccttaaaaggggcaggttcaggggcccggtgctcttcctcccttccatgcacccccatgcccatttgcacagctgcccaggtacccctaacagtggggaggggtcacagggagggggtagcgggaccagtccctgttatctatttaaaaagtgatgatgtaatatattggggtggcggggagatcgggttgtcctgggcctcatcttagcatttcaggtgatggggggagcccagggctggggagacctggggcccagccccagaaagtggggacaatgtggcctcccttctccctactttcggctttcccagtcagtgccttagggggagaggcactccccccctcctattcccttccccccaccccaactcccccacctcgggtgtaagcgacaggaagaaataataataatttaagattca

SEQ ID NO: 15 - DNA, 3003 bp, Homo sapiens
cgaagtcaagacgtctggaaagaattacccagtcctggcttcgagcagcccattgaaccagagacttgaaacagccccagccaaagacttttctcccaattctgcgcttcctgggttctgctgagtcttccacaggctttttttttttttttttttttttaagacgaaaaagagattttctgttatcgggggcagaaagactgaagcacaaaaaaaaaaaaaaagaaaagaaaagaaaagaaaaaagaaaagttaatttatttttaaagcataatttttttaagaattagactgaagtgcaacggaaacataaagagaatattagtgaaattattttttaaagtggggaagaatcaaacatttaagactcccctatcctttttaaatgttgtttttaaatttcttattttttttggccggtcgtctcaaattcatctgatctcttattacctcaattttggaaactgcccgccaccgaccctccgggaccacacagacaggctgaggacgactttatgaccaagagctgaacaagatgcattgtgagaggtttctatgtatcctgagaataattggaaccacactctttggagtctctctcctccttggaatcacagctgcttatattgttggctaccagtttatccaaacggataattactatttctcttttggactgtatggtgcctttttggcatcacacctcatcatccaaagcctgtttgcctttttggagcaccgaaaaatgaaaaaatccctagaaacccccataaagttgaacaaaacagttgccctttgcatcgctgcctatcaagaagatccagactacttaaggaaatgtttgcaatctgtgaaaaggctaacctaccctgggattaaagttgtcatggtcatagatgggaactcagaagatgacctttacatgatggacatcttcagtgaagtcatgggcagagacaaatcagccacttatatctggaagaacaacttccacgaaaagggtcccggtgagacagatgagtcacataaagaaagctcgcaacacgtaacgcaattggtcttgtccaacaaaagtatctgcatcatgcaaaaatggggtggaaaaagagaagtcatgtacacagccttcagagcactgggacgaagtgtggattatgtacaggtttgtgattcagacactatgcttgacccagcctcatctgtggagatggtaaaagttttagaagaagatcccatggttggaggtgttgggggagatgtccagattttaaacaagtacgattcctggatctcattcctcagcagtgtaagatattggatggcttttaatatagaaagggcctgtcagtcttattttgggtgtgttcagtgcattagtggacctctgggaatgtacagaaactccttgttgcatgagtttgtggaagattggtacaatcaagaatttatgggcaaccaatgtagctttggtgatgacaggcatctcacgaaccgggtgctgagcctgggctatgcaacaaaatacacagctcgatctaagtgccttactgaaacacctatagagtatctcagatggctaaaccagcagacccgttggagcaagtcctacttccgagaatggctgtacaatgcaatgtggtttcacaaacatcacttgtggatgacctacgaagcgattatcactggattctttcctttctttctcattgccacagtaatccagctcttctaccggggtaaaatttggaacattctcctcttcttgttaactgtccagctagtaggtctcataaaatcatcttttgccagctgccttagaggaaatatcgtcatggtcttcatgtctctctactcagtgttatacatgtcgagtttacttcccgccaagatgtttgcaattgcaacaataaacaaagctgggtggggcacatcaggaaggaaaaccattgttgttaatttcataggactcattccagtatcagtttggtttacaatcctcctgggtggtgtgattttcaccatttataaggagtctaaaaggccattttcagaatccaaacagacagttctaattgttggaacgttgctctatgcatgctattgggtcatgcttttgacgctgtatgtagttctcatcaataagtgtggcaggcggaagaagggacaacaatatgacatggtgcttgatgtatgatcttccatgttttgacgtttgcagtcacacacaacaccttagttcctctaggggctgtacagtattgtggcatcagataatgccaccaaaggagacatatcactgctgctgggacttgaacaaagacatttatatgggtttattttcattctgccaaagtaaaacaatacatcaacaagaagaaactcagatttaacctgttatttctatgaaaatgggatgaattctttgtttatgcactttttccttactgtgcatccgcctgaaagtgttttggcctatatacctcactagccatgctttatgtgggttatcatggaagaaaaggattttggaaactcaaggaaaagttctttcaacctatacaacctaacttatggactgtttgatagatgataattttttttttttaggaaggattttctttttaactttaccaaatgaaatgccaaaggaagttttaaaggccgtggctgtgctgtatttgatataattgtactgtgtttttaaattgtgtatgccaatcttaaagacaaattttgcatattctctattttacttttctgccaaaataaacctgttcttccttttttaaaataaaataagttcttaaaaaatttatacttaaaaaatcctgcccaaaatgtgaagcttggttgactgatgttcatgatagaaagaataaaatgtttctctctctctaccttttaaaattgaatagtttatttctgtgaaagaagtatttaaactttcaatattttaactttttgtttttatttcttttagaaaaggccaatatacctatcgcg

SEQ ID NO: 16 - DNA, 2516 bp, Homo sapiens
ccttcctccaggagtctctggtgcagctggggtggaatctggccaggccctgcttaggcccccatcctggggtcaggaaatttggaggataaggcccttcagccccaaggtcagcagggacgagcgggcagactggcgggtgtacaggagggctgggttgacctgtccttggtcactgaggccattggatcttcctccagtggctgccaggatttctggtggaagagacaggaaggcctcccccccttggtcgggtcagcctgggggctgagggcctggctgtcagccactcttcccagaacatatgtcatggcctcagtggctcatggggaagcaggggtgggcgagcttaggctagagcaagtcctgtgggagatggcagaggcctggtctgagaggcaactcggatgtgccctccagtggccatgctcccctccatgcgtctcccctgccctcctggagccctgcaggtcaatgtttaacagaaaccagagcagcggtggattaatgcgcaagggctcagccccccagccctgagcagtgggggaatcggagactttgcaacctgttctcagctctgcctcccctggccaggttgtcctcgaccagtcccgtgccatggcagcccacctgcttcccatctgcgccctcttcctgaccttactcgatatggcccaaggctttaggggccccttgctacccaaccggcccttcaccaccgtctggaatgcaaacacccagtggtgcctggagaggcacggtgtggacgtggatgtcagtgtcttcgatgtggtagccaacccagggcagaccttccgcggccctgacatgacaattttctatagctcccagctgggcacctacccctactacacgcccactggggagcctgtgtttggtggtctgccccagaatgccagcctgattgcccacctggcccgcacattccaggacatcctggctgccatacctgctcctgacttctcagggctggcagtcatcgactgggaggcatggcgcccacgctgggccttcaactgggacaccaaggacatttaccggcagcgctcacgggcactggtacaggcacagcaccctgattggccagctcctcaggtggaggcagtagcccaggaccagttccagggagctgcacgggcctggatggcaggcaccctccagctggggcgggcactgcgtcctcgcggcctctggggcttctatggcttccctgactgctacaactatgactttctaagccccaactacaccggccagtgcccatcaggcatccgtgcccaaaatgaccagctagggtggctgtggggccagagccgtgccctctatcccagcatctacatgcccgcagtgctggagggcacagggaagtcacagatgtatgtgcaacaccgtgtggccgaggcattccgtgtggctgtggctgctggtgaccccaatctgccggtgctgccctatgtccagatcttctatgacacgacaaaccactttctgcccctggatgagctggagcacagcctgggggagagtgcggcccagggggcagctggagtggtgctctgggtgagctgggaaaatacaagaaccaaggaatcatgtcaggccatcaaggagtatatggacactacactggggcccttcatcctgaacgtgaccagtggggcccttctctgcagtcaagccctgtgctccggccatggccgctgtgtccgccgcaccagccaccccaaagccctcctcctccttaaccctgccagtttctccatccagctcacgcctggtggtgggcccctgagcctgcggggtgccctctcacttgaagatcaggcacagatggctgtggagttcaaatgtcgatgctaccctggctggcaggcaccgtggtgtgagcggaagagcatgtggtgattggccacacactgagttgcacatattgagaacctaatgcactctgggtctggccagggcttcctcaaatacatgcacagtcatacaagtcatggtcacagtaaagagtacactcagccactgtcacaggcatattccctgcacacacatgcatacttacagactggaatagtggcataaggagttagaaccacagcagacaccattcattccatgtccatatgcatctacttggcaaggtcatagacaattcctccagagacactgagccagtctttgaactgcagcaatcacaaaggctgacattcactgagtgcctactctttgccaatccccgtgctaagcgttttatgtggacttattcattcctcacaatgaggctatgaggaaactgagtcactcacattgagagtaagcacgttgcccaaggttgcacagcaagaaaagggagaagttgagattcaaacccaggctgtctagctccgggggtacagcccttgcactcctactgagtttgtggtaaccagccctgcacgacccctgaatctgctgagaggcaccagtccagcaaataaagcagtcatgattta

SEQ ID NO: 17 - DNA, 19 bp, Artificial Sequence
  RNAi
ttctccgaacgtgtcacgt

SEQ ID NO: 18 - DNA, 19 bp, Artificial Sequence
  RNAi
ggaagtcacagatgtatgt

SEQ ID NO: 19 - protein, 154 aa, Mus musculus
PQASCCEDRVHCCPHGAFCDLVHTRCITPTGTHPLAKKLPAQRTNRAVALSSSSKEDATTDLLTKLPAHTVGDVKCDMEVSCPDGYTCCRLQSGAWCEQGPHQVPWMEKAPAHLSLPDPQALKRDVPCDNVSSCPSSDTCCQLTSGEWGCCPIP

SEQ ID NO: 20 - protein, 24 aa, Opisthorchis viverrini
CPDPVYTCRPGQTCCRGLHGYGCC

SEQ ID NO: 21 - protein, 24 aa, Opisthorchis viverrini
CADPVYTCRPGQTCCRGLHGYGCC

SEQ ID NO: 22 - protein, 24 aa, Opisthorchis viverrini
CPDAVYTCRPGQTCCRGLHGYGCC

SEQ ID NO: 23 - protein, 24 aa, Opisthorchis viverrini
CPDPVYTCRAGQTCCRGLHGYGCC

SEQ ID NO: 24 - protein, 24 aa, Opisthorchis viverrini
CADAVYTCRAGQTCCRGLHGYGCC

SEQ ID NO: 25 - protein, 57 aa, Artificial Sequence
  Generic granulin structure
  misc_feature (1)..(1): Xaa can be any naturally occurring amino acid
  MISC_FEATURE (2)..(3): X at positions 2-3 may be any naturally-occurring amino acid, and up to 1 of them may be absent
  MISC_FEATURE (5)..(6): X at positions 5-6 may be any naturally-occurring amino acid, and up to 1 of them may be absent
  misc_feature (7)..(10): Xaa can be any naturally occurring amino acid
  misc_feature (12)..(16): Xaa can be any naturally occurring amino acid
  misc_feature (19)..(26): Xaa can be any naturally occurring amino acid
  misc_feature (29)..(34): Xaa can be any naturally occurring amino acid
  misc_feature (37)..(41): Xaa can be any naturally occurring amino acid
  misc_feature (44)..(47): Xaa can be any naturally occurring amino acid
  misc_feature (49)..(52): Xaa can be any naturally occurring amino acid
  MISC_FEATURE (53)..(54): X at positions 53-54 may be any naturally-occurring amino acid, and up to 1 of them may be absent
  misc_feature (56)..(57): Xaa can be any naturally occurring amino acid
XXXCXXXXXXCXXXXXCCXXXXXXXXCCXXXXXXCCXXXXXCCXXXXCXXXXXXCXX

SEQ ID NO: 26 - protein, 27 aa, Artificial Sequence
  Generic granulin structure
  misc_feature (1)..(3): Xaa can be any naturally occurring amino acid
  misc_feature (5)..(10): Xaa can be any naturally occurring amino acid
  misc_feature (12)..(16): Xaa can be any naturally occurring amino acid
  misc_feature (19)..(25): Xaa can be any naturally occurring amino acid
XXXCXXXXXXCXXXXXCCXXXXXXXCC